(19) United States
(12) Patent Application Publication
(10) Pub. No.: US 2024/0293337 A1
(43) Pub. Date: Sep. 5, 2024

(54) USE OF DOXERCALCIFEROL TO TREAT AND/OR PREVENT UTERINE FIBROIDS OR POLYCYSTIC OVARY SYNDROME

(71) Applicant: The University of Chicago, Chicago, IL (US)

(72) Inventors: Ayman Al-Hendy, Hinsdale, IL (US); Ana Corachán, Chicago, IL (US); Hang-Soo Park, Chicago, IL (US)

(73) Assignee: The University of Chicago, Chicago, IL (US)

(21) Appl. No.: 18/592,394

(22) Filed: Feb. 29, 2024

Related U.S. Application Data
(60) Provisional application No. 63/488,090, filed on Mar. 2, 2023.

(51) Int. Cl. A61K 31/047

(52) U.S. Cl. CPC A61K 31/047

(57) ABSTRACT

In aspects, the present disclosure provides a method of treating or preventing uterine fibroids (UF) or polycystic ovary syndrome (PCOS) in a female mammal, the method comprising, consisting essentially of, or consisting of administering to the female mammal an effective amount of doxercalciferol. In aspects, the present disclosure provides a method of treating insulin resistance, infertility, obesity, and/or hyperandrogenism related to PCOS in a female mammal.

### CROSS-REFERENCE TO RELATED APPLICATION

This patent application claims the benefit of U.S. Provisional Patent Application No. 63/488,090, filed Mar. 2, 2023, which is incorporated by reference herein in its entirety

### BACKGROUND

Uterine fibroids (UF) are the most common pelvic tumor in women of reproductive age, affecting around 70% of women. Currently surgery, myomectomy or hysterectomy, continues to be the main option for treatment. Hysterectomy is the most invasive yet effective option, being the only one that eliminates the risk of future recurrence; however, it is not a choice for patients who wish to preserve fertility. Approximately 600,000 hysterectomies are performed in the United States per year, UF being the major indication, at an annual cost of $5 billion dollars. Alternative treatments have been developed based on blocking estrogen and progesterone. However, for patients wishing to become pregnant, blocking estrogen and progesterone also causes complications due to side effects including anovulation and infertility.

Polycystic ovarian syndrome (PCOS) is the most common endocrine and metabolic disorder in reproductive-aged women, affecting 4-18% of women. It is a heterogeneous disorder characterized by hyperandrogenism and chronic anovulation. Its clinical features include hirsutism, irregular menses, and infertility. Obesity is also associated with PCOS, and it has been proposed that it could be responsible for insulin resistance and associated hyperinsulinemia in women with PCOS. Obesity is also linked with infertility, and it has been demonstrated that weight loss in women with obesity and PCOS restores normal menstrual cycles and improves fertility rate.

There is an ongoing need in the art for fertility-friendly treatment of UFs and of insulin resistance and infertility in PCOS.

### BRIEF SUMMARY

In aspects, the present disclosure provides a method of treating or preventing uterine fibroids (UF) or polycystic ovary syndrome (PCOS) in a female mammal, the method comprising, consisting essentially of, or consisting of administering to the female mammal an effective amount of doxercalciferol.

Additional aspects of the present disclosure are as described herein.

### BRIEF DESCRIPTION OF THE DRAWINGS

FIGS. 1A-1D show effects of a doxercalciferol treatment protocol on adipocyte morphology. FIG. 1A is a diagram of the protocol for testing 24 hour doxercalciferol treatment on the immortalized adipocyte model using SW872 cells and Mesenchymal Stem Cell (MSC) derived adipocytes. FIG. 1B is a representative micrographs showing the morphology of SW872 cells from the control, 1 nm, 10 nM, and 100 nM doxercalciferol treatment groups after 24 hours, scale bar size is 250 μm. FIG. 1C is a bar graph showing the number of cells in the control, 1 nm, 10 nM, and 100 nM doxercalciferol treatment groups. FIG. 1D is a representative micrographs showing the cell morphology changes during adipocyte differentiation of MDS derived adipocytes from the control, 1 nm, 10 nM, and 100 nM doxercalciferol treatment groups at Day 1, Day 7, Day 14, and Day 14 with oil red-O staining, scale bar size is 250 μm. Data are presented as mean with standard deviation shown as error bars, * indicates a p value <0.05, and NS indicates not significant.

FIGS. 2A-2I show effects of doxercalciferol treatment on adipocyte gene expression. FIG. 2A is a graph showing the relative gene expression results of an ANOVA on adipogenesis gene expression in SW872 cells from the 10 nM doxercalciferol treatment group. FIG. 2B is a volcano plot showing the altered gene expression in SW872 cell after 24 hours of 10 nM doxercalciferol treatment. FIGS. 2C-2I are bar graphs showing the relative gene expression after 10 nM doxercalciferol treatment for the significantly upregulated genes CEBP (FIG. 2C), CEBPD (FIG. 2D), RXRA (FIG. 2E), and the significantly downregulated genes PRDM16 (FIG. 2F), SREBF1 (FIG. 2G), WNT1 (FIG. 2H), and WNT10B (FIG. 2I). Data are presented as mean with standard deviation shown as error bars and * indicates a p value <0.05.

FIGS. 3A-3H show effects of doxercalciferol treatment on adipocyte protein expression. FIG. 3A is an image of an antibody bases obesity related cytokine assay in SW872 secretomes in a control and 10 nM doxercalciferol treatment group after 24 hours.

FIGS. 3B-3H are bar graphs showing the protein expression level in control and 10 nM doxercalciferol treatment groups for the significantly downregulated cytokine production of CXCL10 (FIG. 3B), MIF (FIG. 3C), CCL18 (FIG. 3D), CCL5 (FIG. 3E), TNF RII (FIG. 3F), TIMP-2 (FIG. 3G), and TNF alpha (FIG. 3H). Data are presented as mean with standard deviation shown as error bars and * indicates a p value <0.05. ** indicates a p value <0.005, *** indicates a p value <0.0005, **** indicates a p value <0.0001, and NS indicates not significant

FIGS. 4A-4F show effects of a doxercalciferol treatment protocol on theca-like cells. FIG. 4A is a diagram of the protocol for testing 24 hour doxercalciferol treatment on theca-like cells using H295R cells. FIG. 4B is a representative micrographs showing the morphology of H295R cells from the control, 1 nm, 10 nM, and 100 nM doxercalciferol treatment groups after 24 hours, scale bar size is 250 μm. FIG. 4C is a bar graph showing the number of cells in the control, 1 nm, 10 nM, and 100 nM doxercalciferol treatment groups. FIGS. 4D and 4E are bar graphs showing the relative gene expression of the androgen producing genes CYP11A1 (FIG. 4D), and CYP17A1 (FIG. 4E). FIG. 4F is a bar graph showing the testosterone production level in H295R cell conditioned media. Data are presented as mean with standard deviation shown as error bars and * indicates a p value <0.05. ** indicates a p value <0.005, *** indicates a p value <0.0005, **** indicates a p value<0.0001, and NS indicates not significant.

FIGS. 5A-5E show effects of a doxercalciferol treatment protocol on PCOS-related obesity in mice. FIG. 5A is a diagram of the protocol for testing 24 hour doxercalciferol treatment on letrozole induced polycystic ovary syndrome (PCOS) mouse models. FIG. 5B is a graph showing changes in mouse body weight during the PCOS induction by letrozole. FIG. 5C is a graph showing changes in mouse body weight during the PCOS induction by letrozole and subsequent doxercalciferol treatment. FIG. 5D is a graph showing mouse body weight in control, PCOS, and PCOS with doxercalciferol injection (PCOS+Dox) treatment groups. FIG. 5E is a graph showing blood glucose levels 120 minutes after intraperitoneal (IP) glucose injection in control, PCOS, and PCOS+Dox treatment groups. Data are presented as mean with standard deviation shown as error bars and * indicates a p value <0.05, ** indicates a p value <0.005, *** indicates a p value <0.0005, **** indicates a p value<0.0001, and NS indicates not significant.

FIGS. 6A-6F show effects of doxercalciferol treatment on PCOS-related infertility in mice. FIG. 6A is a graph showing the pregnancy rate of mice from the control, induced PCOS, and induced PCOS with doxercalciferol injection (PCOS+Dox) treatment groups. FIG. 6B is a graph showing the average number of live and dead pups from the control, PCOS, and PCOS+Dox treatment groups. FIG. 6C is a graph showing the total number of live and dead pups from the control, PCOS, and PCOS+Dox treatment groups. FIG. 6D is a graph showing the required time for pregnancy based on delivery date for mice from the control, PCOS, and PCOS+Dox treatment groups. FIG. 6E is a graph showing the average body weight at day 0), day 5, and day 10 of pups from the control, PCOS, and PCOS+Dox treatment groups. FIG. 6F is a graph showing the average body weight change, or growth rate, at day 0 day 5, and day 10 of pups from the control, PCOS, and PCOS+Dox treatment groups. Data are presented as mean with standard deviation shown as error bars and * indicates a p value <0.05, ** indicates a p value <0.005, and *** indicates a p value <0.0001, **** indicates a p value<0.0001, and NS indicates not significant.

FIGS. 7A-7B show effects of doxercalciferol treatment on cell morphology in PCOS mice. FIGS. 7A-7B are representative micrographs from mice in the control, induced PCOS, and induced PCOS with doxercalciferol injection (PCOS+Dox) treatment groups stained with hematoxylin and eosin (H&E) showing tissue from the ovary, uterus, and gonad white fat (FIG. 7A), and the kidney and liver (FIG. 7B). Scale bar size is 500 μm.

FIGS. 8A-8I show the effects of doxercalciferol treatment on serum levels in PCOS mice. FIGS. 8A-8I are bar graphs showing serum levels of BUN (FIG. 8A), ALT (FIG. 8B), ALP (FIG. 8C), AST (FIG. 8D), TBIL (FIG. 8E), Calcium (FIG. 8F), TP (FIG. 8G), ALB (FIG. 8H), and NA+ (FIG. 8I) for mice in the control, induced PCOS, and induced PCOS with doxercalciferol injection (PCOS+Dox) treatment groups, and the reference ranges for C57BL/6 strain mice.

FIGS. 9A-9F show effects of doxercalciferol treatment on tumors, weight, and glucose levels in patient derived xenograft (PDX) mouse model of uterine fibroids (UF). FIG. 9A is a diagram of the protocol for testing 24 hour doxercalciferol treatment on the patient derived xenograft (PDX) mouse model of uterine fibroids (UF). FIGS. 9B and 9C are graphs showing the percentage of tumor growth in PDX mice over 6 weeks in control (FIG. 9B) and doxercalciferol (FIG. 9C) treatment groups. FIG. 9D is a graph showing the percent weight change in PDX mice from the control and doxercalciferol treatment groups over 6 weeks. FIGS. 9E and 9F are graphs showing the glucose (FIG. 9E) and fasting glucose (FIG. 9F) levels of control and doxercalciferol treated PDX mice. Data are presented as mean with standard deviation shown as error bars and * indicates a p value <0.05, **** indicates a p value<0.0001, and NS indicates not significant.

FIGS. 10A-10F show effects of doxercalciferol treatment on liver enzyme levels, AMH levels, and ovarian follicle development in the PDX mouse model of UF. FIGS. 10A-10C are graphs showing the level of the liver enzymes AST (FIG. 10A), ALT (FIG. 10B), and total bilirubin (FIG. 10C) in PDX mice from the control and doxercalciferol treatment groups. FIG. 10D is a graph showing the AMH serum levels in PDX mice from the control and doxercalciferol treatment groups. FIG. 10E is a graph showing the number of follicles per section in ovary tissue slices of PDX mice from the control and doxercalciferol treatment groups. FIG. 10F is a graph showing the number of primordial, primary, secondary, and preantral follicles per section in ovary tissue slices of PDX mice from the control and doxercalciferol treatment groups. Data are presented as mean with standard deviation shown as error bars and NS indicates not significant.

### DETAILED DESCRIPTION

In aspects, the present disclosure provides a method of treating or preventing uterine fibroids (UF) or polycystic ovary syndrome (PCOS) in a female mammal, the method comprising, consisting essentially of, or consisting of administering to the female mammal an effective amount of doxercalciferol.

Doxercalciferol can be administered as transdermal, subcutaneous, topical, oral, absorption through epithelial or mucocutaneous linings, intravenous, intra-ovarian, intranasal, intra-arterial, intramuscular, intratumoral, peritumoral, interperitoneal, intrathecal, rectal, or vaginal formulations. In aspects, doxercalciferol is administered intravenously.

The active agent may be made into an injectable formulation. The requirements for effective pharmaceutical carriers for injectable compositions are well known to those of ordinary skill in the art. In aspects, the active agent, doxercalciferol, is suspended or resuspended (reconstituted) in normal saline, either alone or in combination.

The terms “treat,” “treating,” “treatment,” “therapeutically effective,” “prevention,” etc. used herein do not necessarily imply 100% or complete treatment/prevention/etc. Rather, there are varying degrees, which one of ordinary skill in the art recognizes as having a potential benefit or therapeutic effect. In this respect, the doxercalciferol and methods can provide any amount of any level of treatment or prevention. Furthermore, the treatment/prevention provided by the disclosed method can include the treatment/prevention of one or more conditions or symptoms of the disease or condition being treated/prevented.

In aspects, the method is a method of treating or preventing UFs. In aspects, the method is a method of treating. In aspects, the method is a method of preventing.

As used herein, a “uterine fibroid” (UF), is a benign tumor of the uterus that consists of a mass or population of smooth muscle cells and connective tissue that grows, usually slowly, within the uterine wall. Epidemiologic studies demonstrate that UFs, also known as leiomyomas, initially form after menarche. It is suspected that fibroid growth is due to a monoclonal, deregulated proliferation of uterine smooth muscle myometrial cells. The primary tumor cell type resulting from the growth of the fibroid are derived from myometrial cells.

UFs have a high accumulative incidence. UFs are one of the most common tumors. Complications arising from uterine fibroids account for approximately a third of all hysterectomies performed in the U.S., and are associated with high morbidity from uterine bleeding and pain. By age 50 approximately 75% of women have developed UFs. A significant number of those with UFs suffer from debilitating pelvic pain, heavy and prolonged bleeding, which may lead to anemia and iron deficiency, bowel and bladder dysfunction, and infertility. UFs also cause symptoms such as low back pain, urinary frequency and urgency, pain during intercourse (dyspareunia), can cause pre-term labor, and have a negative impact on fertility (due to cavity distension, and alteration of endometrial receptivity and sexual function).

In aspects, the method is a method of treating or preventing PCOS. In aspects, the method is a method of treating. In aspects, the method is a method of preventing.

In aspects, the method is a method of treating or preventing PCOS-related infertility. Treating PCOS can involve restoring viability of eggs or ovaries, or can involve restoring fertility. Restoration of fertility may be to any level of fertility greater than experienced with PCOS. Factors indicating greater fertility include, but are not limited to, increased ovarian follicle number, restored estrus cycle, increased pregnancy rate, increased number of offspring, and restored serum hormone aberration. In aspects, treating PCOS involves restoring viability of eggs or ovaries. In aspects, treating PCOS involves restoring fertility. In aspects, fertility is restored to any level of fertility greater than that experienced with PCOS, wherein fertility is determined by number of follicles, pregnancy rate, or delivery rate.

In aspects, the method is a method of treating or preventing PCOS-related insulin resistance. Treating PCOS can involve restoring insulin resistance. Restoration of insulin resistance may be to any level of insulin resistance greater than experienced with PCOS. Factors indicating greater insulin resistance include, but are not limited to, lowered blood glucose levels, reduced obesity, and increased hormone functionality.

In aspects, the method is a method of treating or preventing PCOS-related obesity. Treating PCOS can involve reducing obesity. Reduced obesity may be to any weight lower than experienced with PCOS. Factors indicating reduced obesity include, but are not limited to, reduced weight and reduced body mass index.

In aspects, the method is a method of treating or preventing PCOS-related hyperandrogenism. Treating PCOS can involve reducing androgen levels. Reducing androgen levels may be to any level lower than that experienced with PCOS. Factors indicating reduced androgen levels include, but are not limited to, lowered testosterone levels, lowered androsterone levels, lowered androstenedione levels, reduced hirsutism, reduced acne, reduced androgenic alopecia, and reduced hypermenorrhea.

The disclosed methods comprise using an effective amount of doxercalciferol. An “effective amount” means an amount sufficient to show a meaningful benefit. A meaningful benefit includes, for example, detectably treating, relieving, or lessening one or more symptoms of UFs or PCOS: inhibiting, arresting development, preventing, or halting further development of UFs or PCOS: reducing the severity of UFs or PCOS: preventing UFs or PCOS from occurring in a subject at risk thereof but yet to be diagnosed: reducing the size and/or mass of UFs. The meaningful benefit observed can be to any suitable degree (10, 20, 30, 40, 50, 60, 70, 80, 90% or more). In aspects, one or more symptoms are prevented, reduced, halted, or eliminated subsequent to administration of doxercalciferol described herein, thereby effectively treating the disease to at least some degree.

One skilled in the art will recognize that dosage will depend upon a variety of factors, including the age, condition or disease state, predisposition to disease, genetic defect or defects, and body weight of the subject. The size of the dose will also be determined by the route, timing and frequency of administration as well as the existence, nature, and extent of any adverse side-effects that might accompany the administration of a particular active agent and the desired effect. It will be appreciated by one of skill in the art that various conditions or disease states may require prolonged treatment involving multiple administrations.

The amount (e.g., therapeutically effective amount) of doxercalciferol suitable for administration depends on, e.g., the particular route of administration and the weight of the mammal to be treated. In aspects, the amount of doxercalciferol can be about 300 ng/kg in mice and about 4000 IU in humans, or 0.5 to 2.5 μg per day in human. Several doses can be provided over a period of days or weeks. In aspects, doses can be provided daily for 6 weeks in mice, or 3 times a week for 12 weeks in humans.

The mammal may be any suitable mammal. Mammals include, but are not limited to, the order Rodentia, such as mice, and the order Lagomorpha, such as rabbits. The mammal can be from the order Carnivora, including Felines (cats) and Canines (dogs). The mammal can be from the order Artiodactyla, including Bovines (cows) and Swines (pigs) or of the order Perissodactyla, including Equines (horses). The mammal can be of the order Primates, Cebids, or Simioids (monkeys) or of the order Anthropoids (humans and apes). In aspects, the mammal is human.

The following includes certain aspects of the disclosure.
1. A method of treating or preventing uterine fibroids (UF) or polycystic ovary syndrome (PCOS) in a female mammal, the method comprising, consisting essentially of, or consisting of administering to the female mammal an effective amount of doxercalciferol.
2. The method of aspect 1, wherein the method is a method of treating or preventing UF.
3. The method of aspect 1, wherein the method is a method of treating or preventing PCOS.
4. The method of aspect 3, wherein the method is a method of treating or preventing PCOS-related insulin resistance.
5. The method of aspect 3, wherein the method is a method of treating or preventing PCOS-related infertility.
6. The method of aspect 3, wherein the method is a method of treating or preventing PCOS-related obesity.
7. The method of aspect 3, wherein the method is a method of treating or preventing PCOS-related hyperandrogenism.
8. The method of any one of aspects 1-7, wherein the administration is intravenous.
9 The method of any one of aspects 1-7, wherein the administration is oral.
10. The method of any one of aspects 1-9, wherein the female mammal is human.
11. The method of aspect 10, wherein the effective amount of doxercalciferol is 4000 IU administered 3 times a week for 12 weeks.

It shall be noted that the preceding are merely examples of aspects of the disclosure. Other exemplary aspects are apparent from the entirety of the description herein. It will also be understood by one of ordinary skill in the art that each of these aspects may be used in various combinations with the other aspects provided herein.

The following examples further illustrate aspects of the disclosure, but, of course, should not be construed as in any way limiting its scope.

### Example 1

### Materials and Methods

### Human Liposarcoma Culture

Human liposarcoma SW872 are used as an in vitro cell culture model of adipocytes to assess adipocyte proliferation. SW872 cells are purchased from (ATCC). These cells are isolated from a surgical specimen of a fibrosarcoma removed from a 36 year old male Caucasian donor. The cells are cultured per the recommended guidelines. The cells are subcultured at a ratio of 1:4, resulting in a cell density of approximately 1×10⁶ in T75 flasks.

### Human Mesenchymal Stem Cell (MSC) Derived Adipocyte Culture

Human mesenchymal stem cells (MSC) are used as an in vitro cell culture model of adipocyte precursors to assess adipocyte differentiation. MSC cells are purchased from (Lonza). The cells are cultured in the DMEM/F12 media containing 10% FBS. At approximately 80% confluence, the cells are trypsinized using TrypLE select enzyme (Gibco, MA) and are serially expanded. At the end of the culture expansion, MSCs are collected and centrifuged at 300×g for 5 minutes.

Treatment of Human MSC Derived Adipocytes with Doxercalciferol

Human MSCs are treated with doxercalciferol and are cultured in adipocyte induction medium for 14 days. Mesenchymal Adipogenesis Kit (Sigma) and manufacturer's induction protocol are used for adipocyte induction. Imaging and morphology analysis is performed on days 1, 7, 10, and 14. On day 14 when adipocyte differentiation is completed, the cells undergo oil red-O staining and are imaged for further morphology analysis.

Treatment of SW872 Cells with Doxercalciferol

SW872 cells are subcultured in T75 flasks for 24 hours. Cells are then treated for 24 hours with serum free basal media as a control or doxercalciferol at 1 nM, 10 nM, or 100 nM diluted in basal media (serum-free). After the incubation period, cells are imaged and analyzed for morphology and cell count. Cells are then collected for analysis of adipogenesis-related gene expression and cell culture media are collected for analysis of obesity related cytokines.

Quantitative Real-Time PCR (qRT-PCR)

RNA is extracted from SW872 cells treated with doxercalciferol. RNA extraction is done using TRIzol (Invitrogen, USA) according to the manufacturer's instructions. The concentration and purity of the extracted RNA is checked using a NanoDrop spectrometer (Thermo Scientific, MA, USA). Quantitative real-time PCR (qPCR) of adipogenesis related genes in SW872 cells is performed using a RT² Profiler™ PCR Array Human Apidogenesis according to the manufacturer's instructions (Qiagen, Sciences Inc., MD, USA).

### Cytokine Analysis

Cell culture media are collected from SW872 cells after 24 hours of 10 nM doxercalciferol treatment. The media is analyzed for obesity related cytokines using a membrane-based antibody array (Ray Biotech, GA, USA) per the manufacturer's protocol, and then quantified using a multimode microplate reader system, Varioskan LUX Multimode Microplate Reader (Thermo Fisher).

### Statistical Analysis

Comparisons between groups are made by ANOVA or Student's t-tests. All data are presented as mean±standard deviation (SD). A difference between groups with a p value <0.05 (*), p value <0.005 (**), p value <0.0005 (*), or p value <0.0001 (****) are considered statistically significant, while those with a p value >0.05 (NS) are considered not significant.

### Results

### Effect of Doxercalciferol Treatment on Immortalized Adipocyte Model (SW872) and Mesenchymal Stem Cell (MSC) Derived Adipocyte

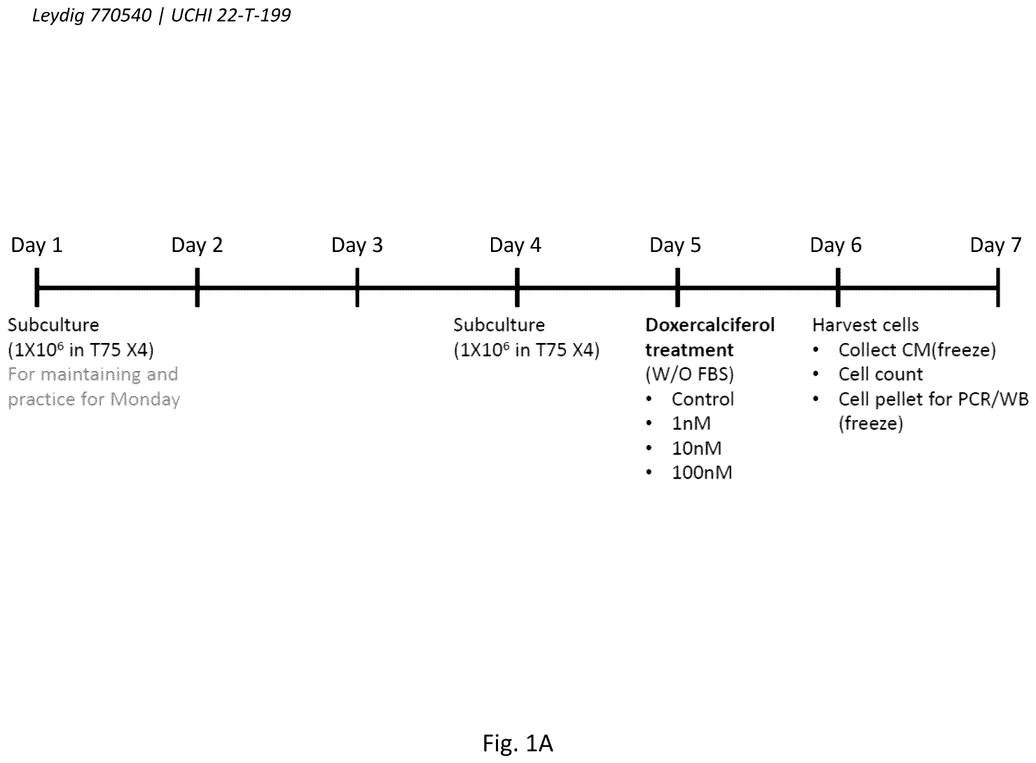

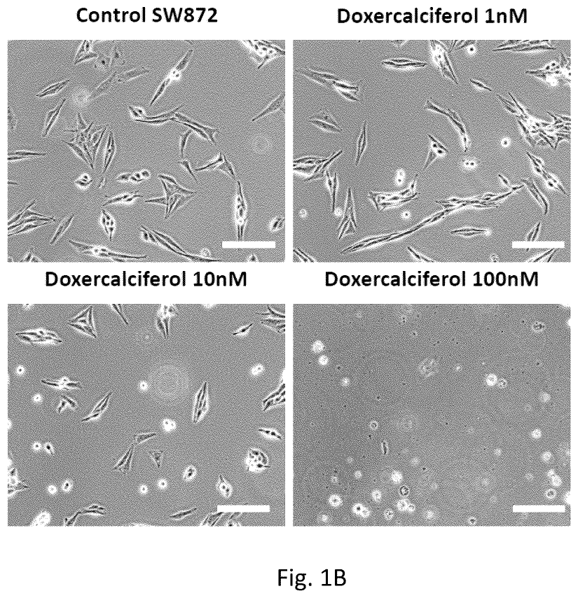

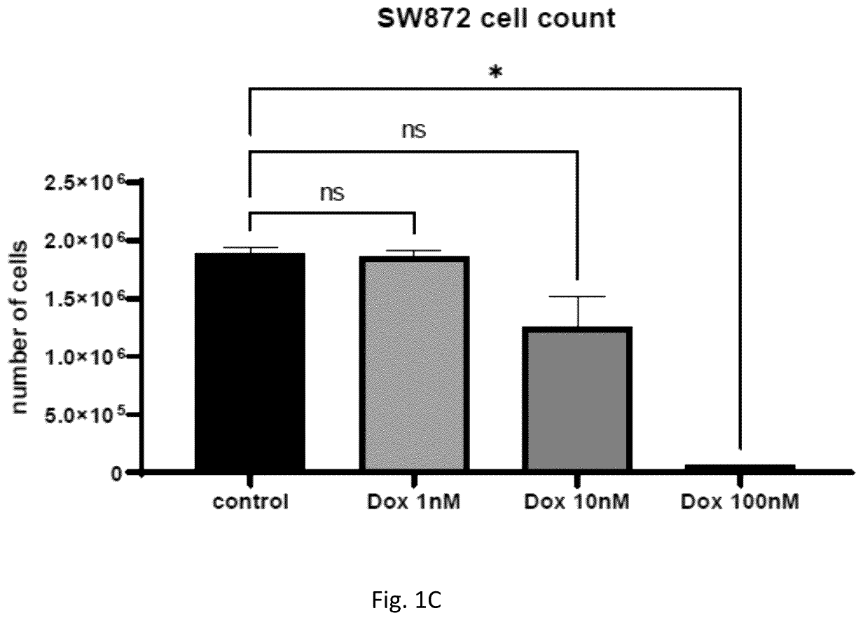

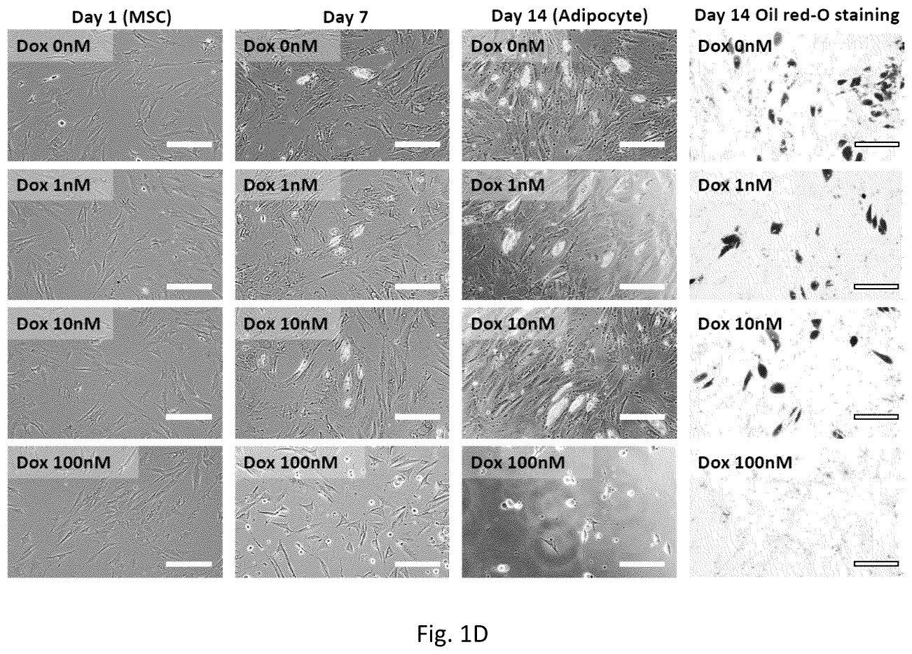

SW872 cells, an immortalized adipocyte model, are incubated with doxercalciferol at 1 nM, 10 nM, or 100 nM doxercalciferol to evaluate therapeutic potential according to the protocol in FIG. 1A. After 24 hours, cell morphology (FIG. 1B) and cell number (FIG. 1C) show a significant reduction in cells in the 100 nM doxercalciferol treatment group, with most cells being dead. MSC derived adipocytes are also evaluated for therapeutic potential and are incubated with 1 nM, 10 nM, or 100 nM doxercalciferol for 14 days. The MSC derived adipocytes show similar results with most cells dead in the 100 nM doxercalciferol treatment group. Oil red-O staining, demonstrates the presence of adipocytes in all treatment groups except the 100 nM group (FIG. 1D). These results suggest doxercalciferol treatment inhibits both adipocyte differentiation and proliferation.

### Effect of Doxercalciferol Treatment on Immortalized Adipocyte Model (SW872) Gene Expression

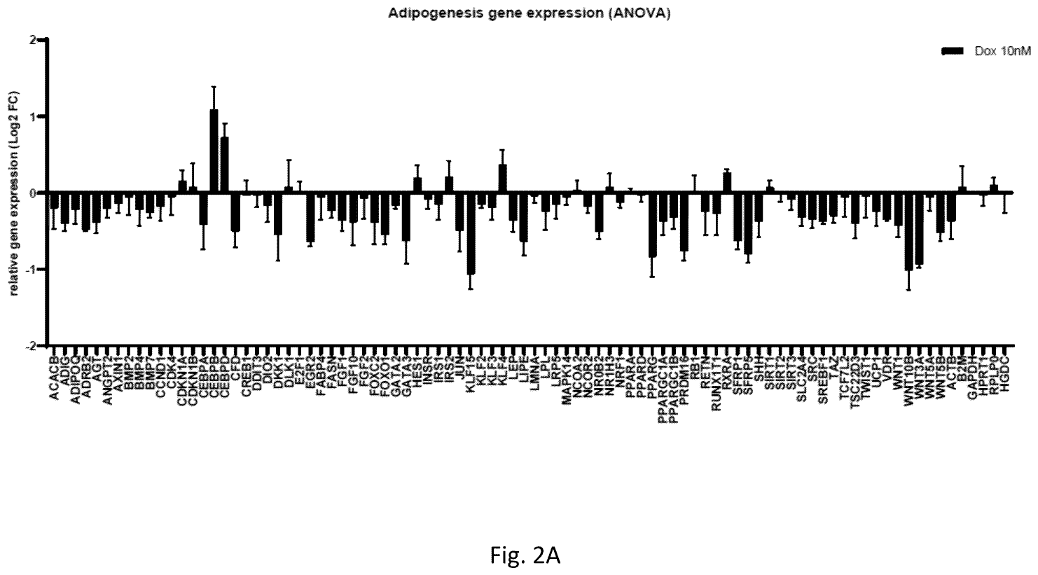

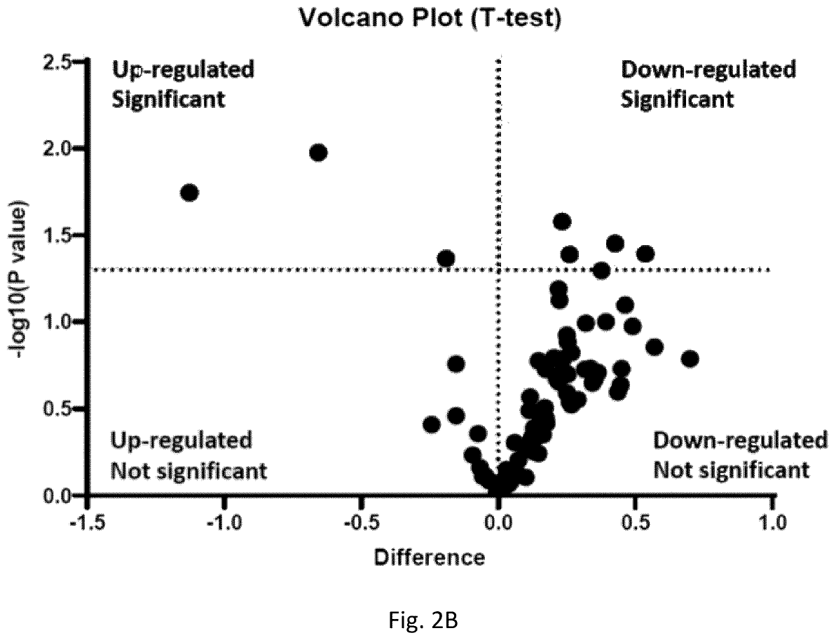

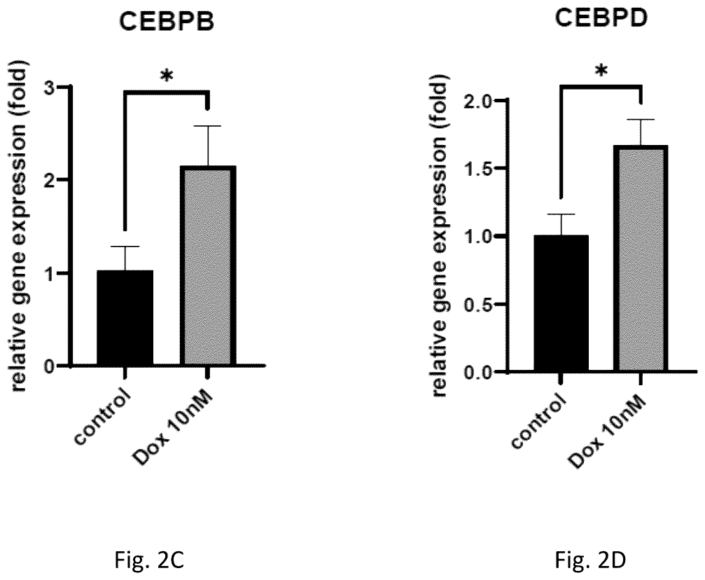

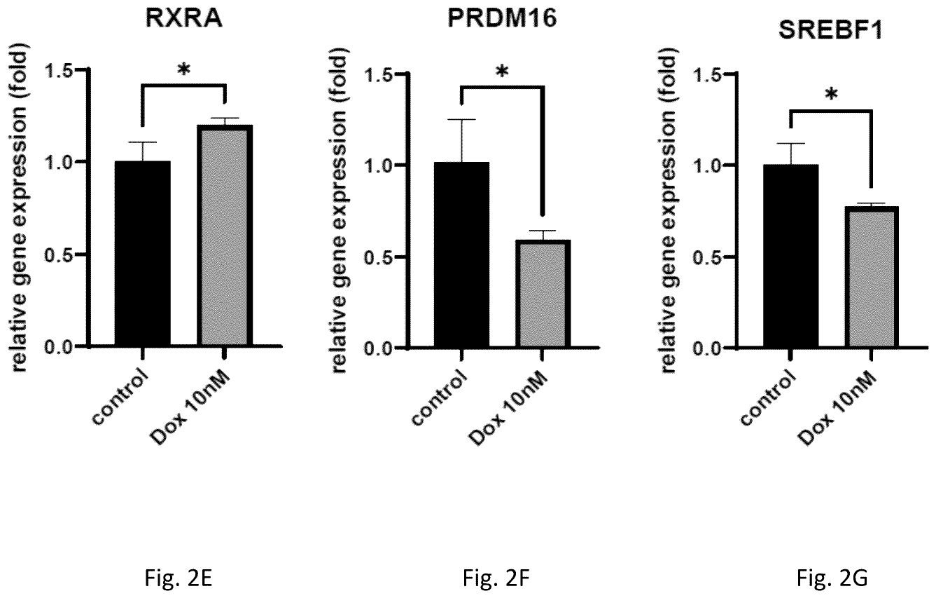

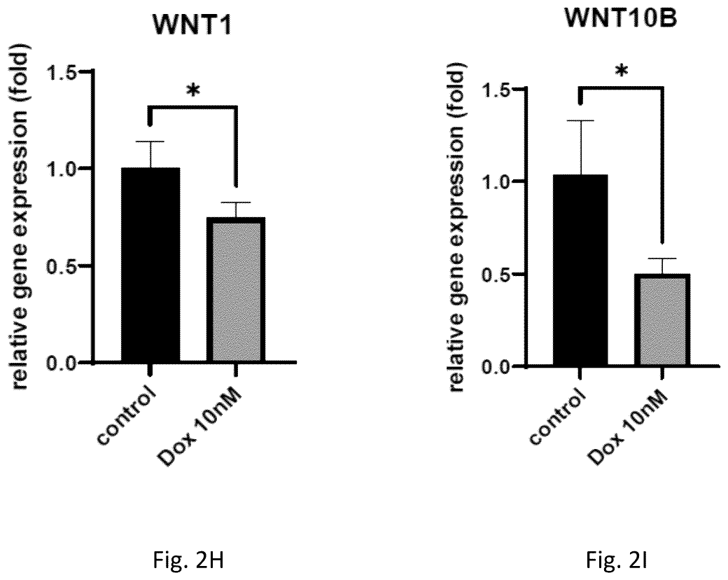

To further investigate the effect of doxercalciferol treatment on SW872 cells an ANOVA is performed on adipogenesis gene expression in SW872 cells from the 10 nM doxercalciferol treatment group. Most adipogenesis genes have their expression downregulated in the presence of doxercalciferol (FIG. 2A), although the downregulation is not significant for most genes (FIG. 2B). The genes that have their expression significantly upregulated included CEBP (FIG. 2C), CEBPD (FIG. 2D), RXRA (FIG. 2E) and those that are significantly downregulated included PRDM16 (FIG. 2F), SREBF1 (FIG. 2G), WNT1 (FIG. 2H), and WNT10B (FIG. 2I). Increased CEBPB expression is known to be related to increase insulin levels and obesity prevention, while increased CEBPD expression is related to lower inflammation levels in obese mice, and increased RXRA expression is related to reduced insulin resistance. These results suggest that doxercalciferol causes the upregulation of genes involved in preventing obesity and obesity related complications.

### Effect of Doxercalciferol Treatment on Immortalized Adipocyte Model (SW872) Cytokine Secretion

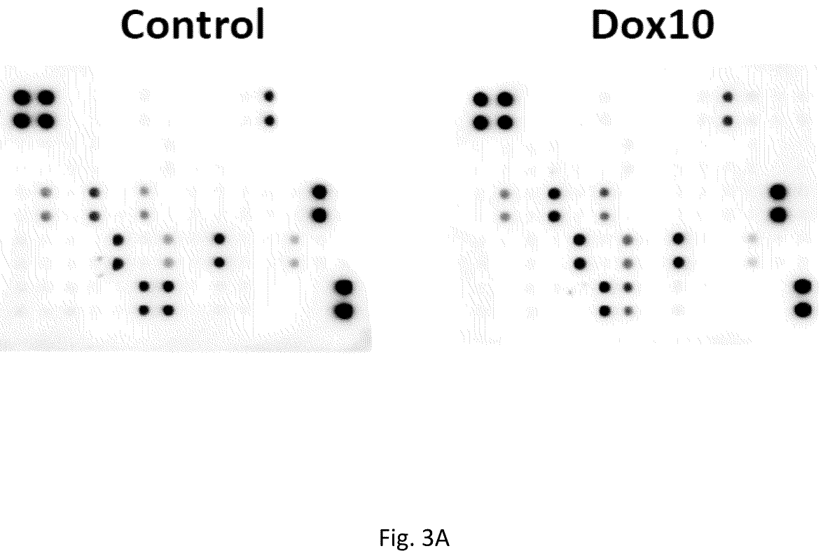

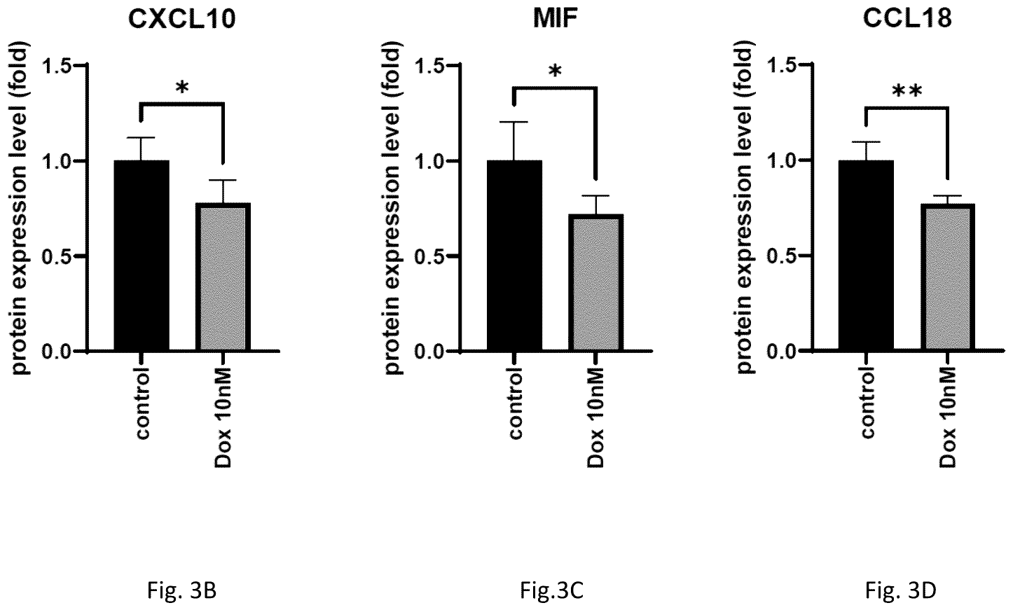

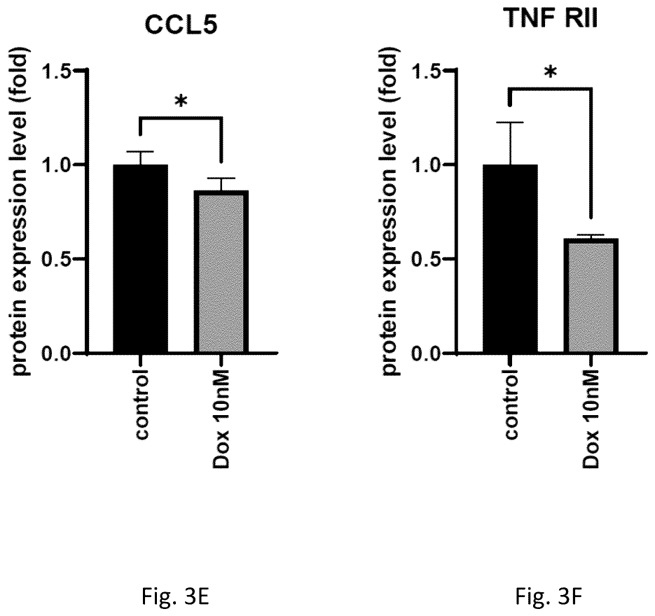

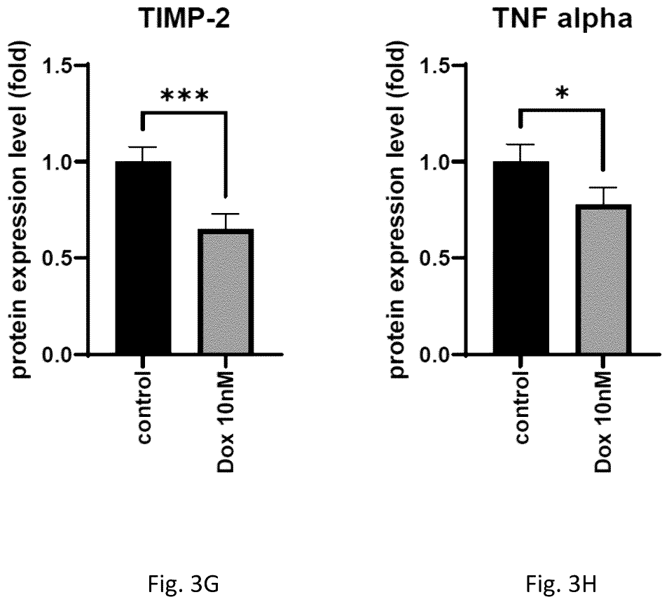

To investigate the effects of doxercalciferol treatment on the cytokine secretions of SW872 cells an antibody obesity related cytokine assay of SW872 secretomes is performed after 24 hours of 10 nM doxercalciferol treatment (FIG. 3A). The results show that doxercalciferol treatment significantly downregulates production of the cytokines of CXCL10 (FIG. 3B), MIF (FIG. 3C), CCL18 (FIG. 3D), CCL5 (FIG. 3E), TNF RII (FIG. 3F), TIMP-2 (FIG. 3G), and TNF alpha (FIG. 3H). These results suggest that doxercalciferol treatment reduces cytokine secretion by adipocytes.

### Example 2

### Materials and Methods

### Human Adrenocortical-Carcinoma Cell Line Culture

Human adrenocortical-carcinoma cells (H295R cells) are used as an in vitro cell culture model of ovarian theca cells to assess androgen production. H295R cells are purchased from ATCC (Manassas, VA, USA, cat. no. ATCC® CRL-2128™) and cultured per the recommended guidelines. These cells are isolated from a 48 year old female donor. Briefly, H295R cells are cultured in flasks pre-coated with extracellular matrix (Gibco, USA, cat. no. S-006-100) with DMEM/F12 (Gibco, cat. no. 21041025) and 2.5% Nu-Serum (Corning, USA). The cells are subcultured at a ratio of 1:4, resulting in a cell density of approximately 3×10⁶ in T75 flasks.

Treatment of H295R Cells with Doxercalciferol

H295R cells are subcultured in T75 flasks for 24 hours. Cells are then treated for 24 hours with serum free basal media as a control or doxercalciferol at 1 nM, 10 nM, or 100 nM diluted in basal media (serum-free) at a 1:1 ratio. After the incubation period, cells are imaged and analyzed for morphology and cell count. Cells are then collected for analysis of steroidogenesis-related gene expression. Cell culture media are collected and used for testosterone quantification using ELISA based Testosterone Parameter Assay Kit (R&D system) per the manufacturer's protocol. The testosterone level is quantified using a multimode microplate reader system, Varioskan LUX Multimode Microplate Reader (Thermo Fisher).

Quantitative Real-Time PCR (qRT-PCR)

RNA is extracted from H295R cells treated with doxercalciferol. RNA extraction is done using TRIzol (Invitrogen, USA) according to the manufacturer's instructions. The concentration and purity of the extracted RNA are checked using a NanoDrop spectrometer (Thermo Scientific, MA, USA). For qPCR of steroidogenesis related genes in H295R cells 1 μg of total RNA is reverse transcribed using RNA to cDNA EcoDry™ Premix (Double Primed) (Takara Bio USA Inc., CA, USA). The reaction mixture is incubated for 1 hour at 42° C.; incubation is stopped at 70° C. for 10 min. qPCR is performed using the CFX96 PCR instrument and SYBR Green Supermix (Bio-Rad, Hercules, CA, USA) with specific primers (RT² Profiler PCR Array for Human Adipogenesis, PAHS-049ZA, QIAGEN) to the target genes in a 20 μL final reaction volume. The delta-delta threshold cycle (ΔΔCt) method is used to calculate the fold change expression in mRNA level in the samples.

### Statistical Analysis

Comparisons between groups are made as described in example 1.

### Results

### Effect of Doxercalciferol Treatment on Theca-Like Cells (H295R) Androgen Production

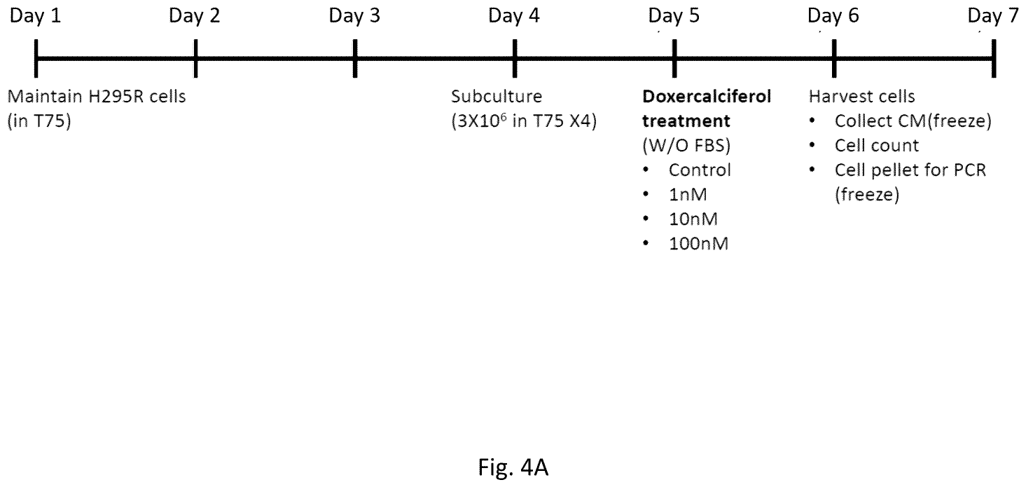

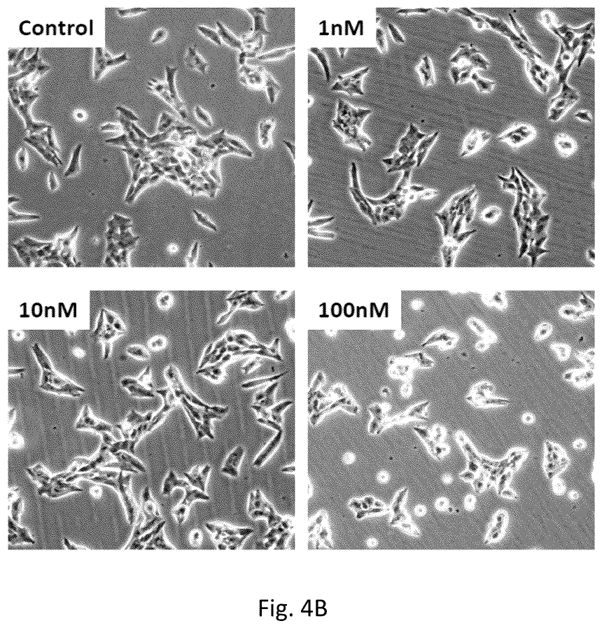

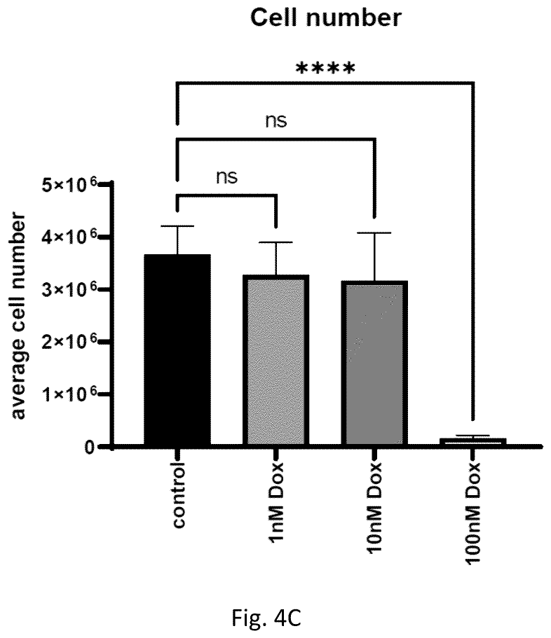

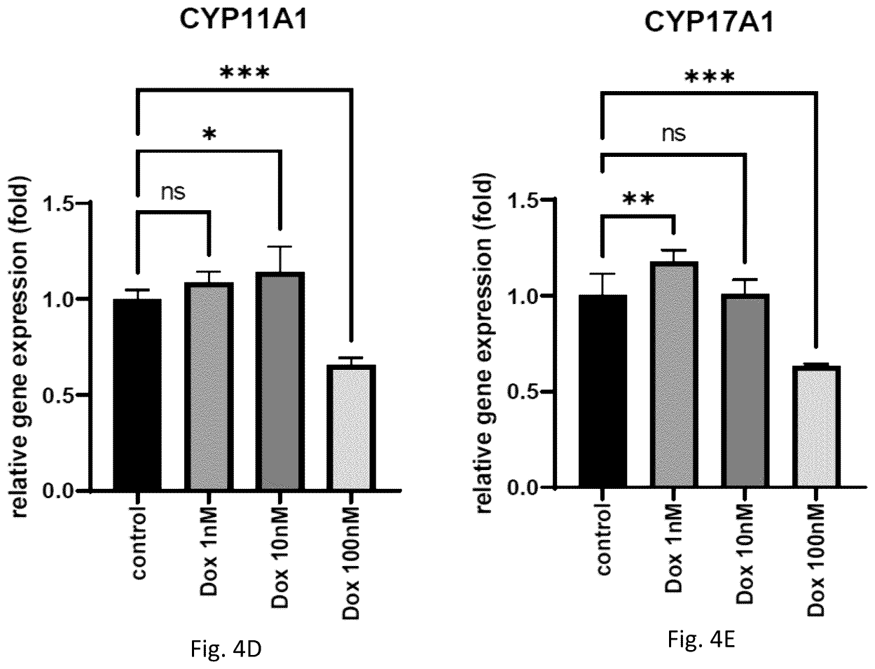

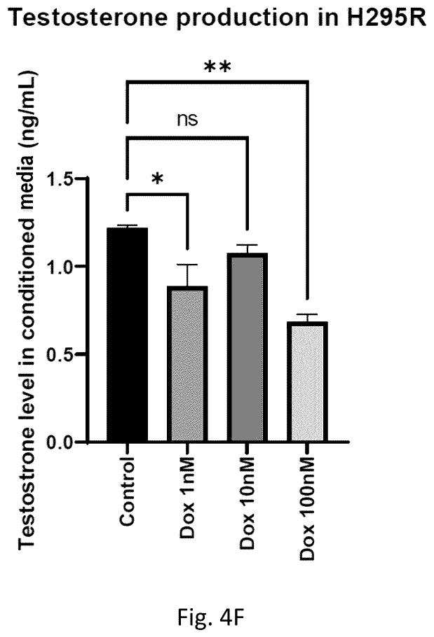

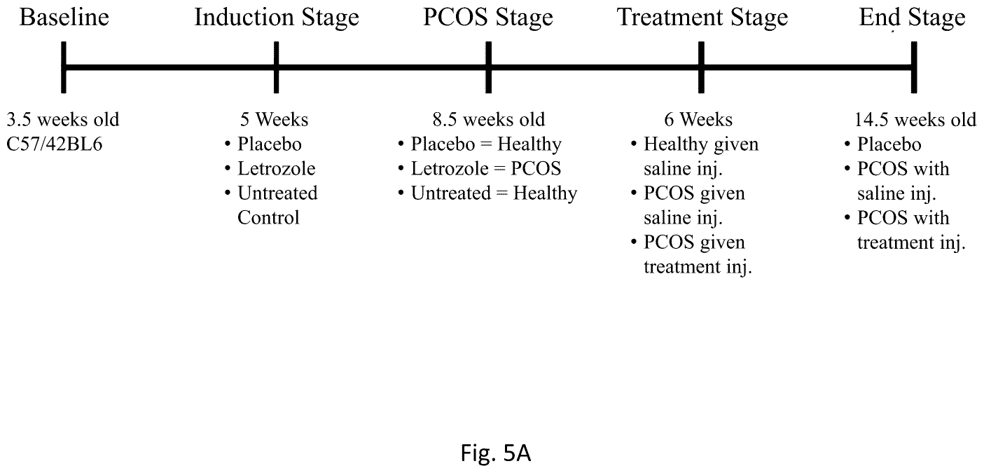

H295R cells are incubated with doxercalciferol at 1 nM, 10 nM, or 100 nM doxercalciferol to evaluate therapeutic potential according to the protocol in FIG. 4A. After 24 hours cell morphology is assessed and most of the cells in the 100 nM doxercalciferol treatment group are dead (FIG. 4B). The graph in FIG. 4C shows that the 100 nM doxercalciferol treatment group exhibit significantly fewer cells than the other groups. Doxercalciferol treatment alters the gene expression of the androgen producing genes CYP11A1 and CYP17A1 in H295R cells. CYP11A1 expression is not significantly changed at 1 nM, but is significantly increased at 10 nM, and is significantly decreased at 100 nM (FIG. 4D). CYP17A1 gene expression is significantly increased at 1 nM, not significantly changed at 10 nM, and significantly decreased at 100 nM (FIG. 4E). Testosterone production of H295R cells is significantly decreased after doxercalciferol treatments at 1 nM and 100 nM, but not significantly changed at 10 nM (FIG. 4F). These results suggest that doxercalciferol treatment prevents theca-like cell proliferation and decreases androgen production of theca-like cells.

### Example 3

### Materials and Methods

### PCOS Mouse Model Doxercalciferol Treatment

2.5-week-old female C57BL/6 mice (Charles River, MA, USA) are housed in a vivarium for 1 week under specific pathogen-free conditions. The animal experiment protocol for this study is approved, and all animal experiments are performed in compliance with policies and guidelines for use of laboratory animals.

At 3.5 weeks of age, mice (n=11/group) are subcutaneously implanted with a placebo or 5 mg letrozole (LTZ) pellet (Innovative Research of America, Sarasota, FL, USA), which provides a constant release of LTZ (50 μg/day). Body weight is monitored weekly before and post-implantation. Body weight and insulin resistance (measured by glucose tolerance test, GTT) are used to monitor development of PCOS characteristics.

Five weeks after placebo or LTZ pellet implantation, mice undergo intraperitoneal injection of doxercalciferol (300 ng/kg) or saline 3 times per week for 6 weeks. Six weeks after doxercalciferol treatment, the mice are anesthetized and ovary, uterus, gonadal white fat, kidney, and liver tissue are collected. Each of these tissues are fixed in 4% paraformaldehyde for further histological analysis.

### Glucose Tolerance Test

Glucose tolerance testing is performed on healthy, PCOS, and treated mice 3 weeks after doxercalciferol treatment. Mice are fasting for 16 h (5 p.m. to 9 a.m.), with free access to drinking water, after which they receive an intra-peritoneal (i.p.) injection of D-glucose (2.0 g/kg body weight). Blood glucose level is measured at 0, 15, 30, 60, 90, and 120 minutes following glucose injection using a Bayer glucose monitor (Roche Diagnostics Corp, IN, USA).

### Breeding Experiments

After doxercalciferol treatment, 6 mice per group from the PCOS experiments are randomly selected for the breeding experiment. One male C57BL/6 breeder mouse is used for every two female mice. The male and female mice are caged together for 10 days. Mating is determined by the presence of sperm plug in the vagina. The pregnancy rate, required time for pregnancy, average number of pups, total live and dead pups, and mouse pup weight and growth rate at birth and days 5 and 10 postpartum are measured.

### Histology and Immunohistochemistry

Ovary, uterus, gonadal white fat, kidney, and liver tissue are collected and fixed in 4% paraformaldehyde. Tissue sections are stained with hematoxylin-eosin (H&E). Histological analyses are performed using Asperio ImageScope (Leica Biosystem, Wetzlar, Germany).

### Serum Measurements

Blood is collected from all groups by cardiac exsanguination under isoflurane anesthesia: serum is separated and stored at −80° C. Serum chemistry analysis including BUN, ALT, ALP, AST, TBIL, calcium, TP, ALB, and NA+ is performed in University of Chicago Animal Resource Center (UC ARC) with VETSCAN® VS2 Chemistry Analyzer (Zoetis).

### Statistical Analysis

Comparisons between groups are made as described in example 1.

### Results

### Effect of Doxercalciferol Treatment on PCOS Mouse Weight and Blood Glucose

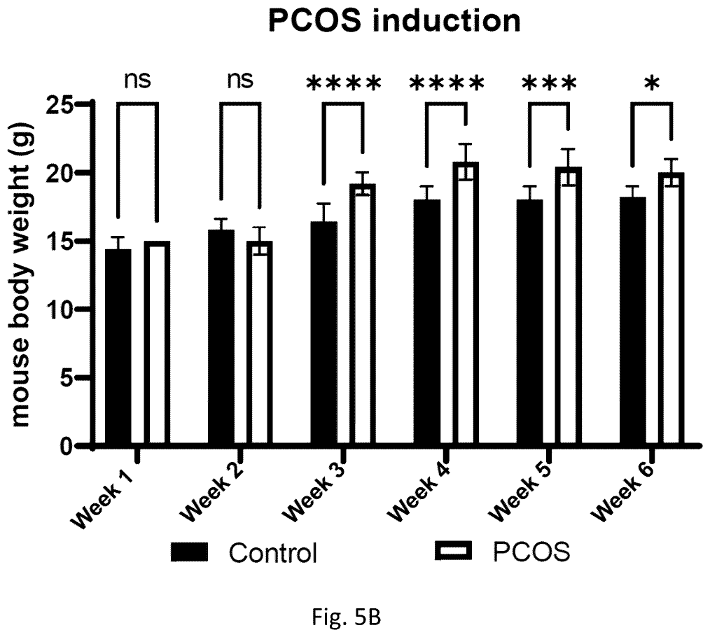

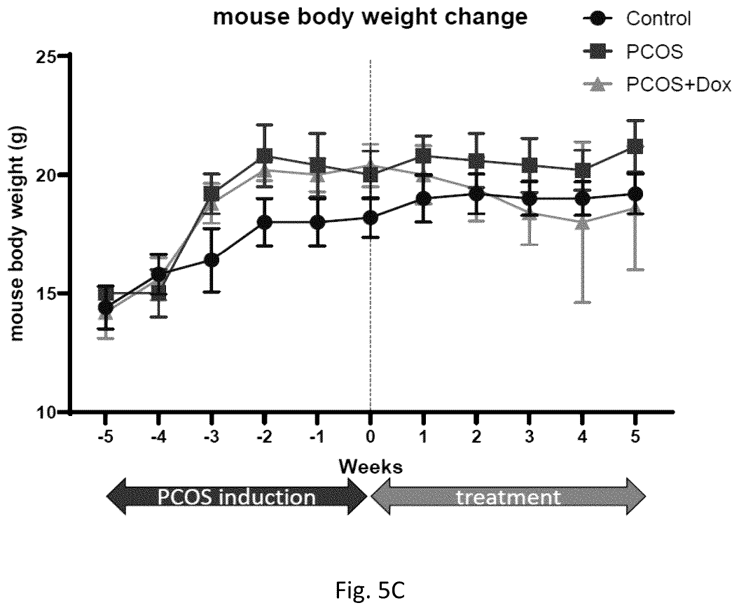

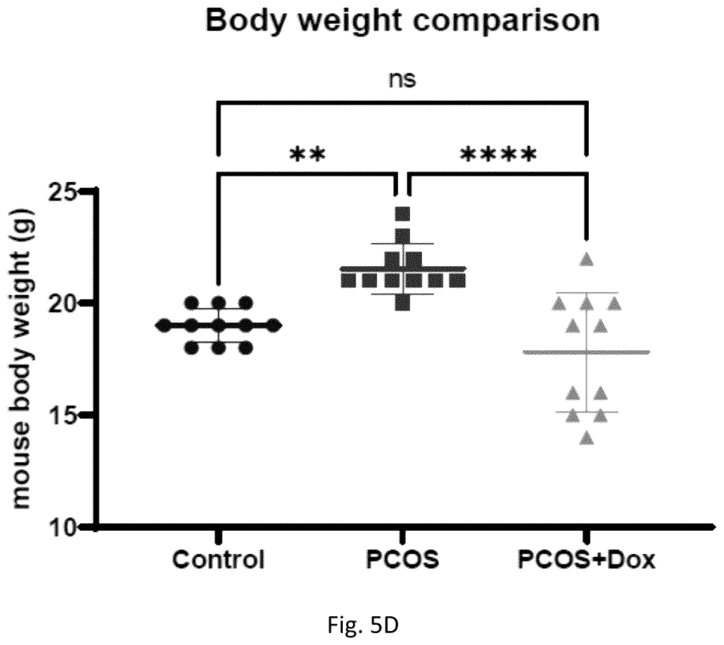

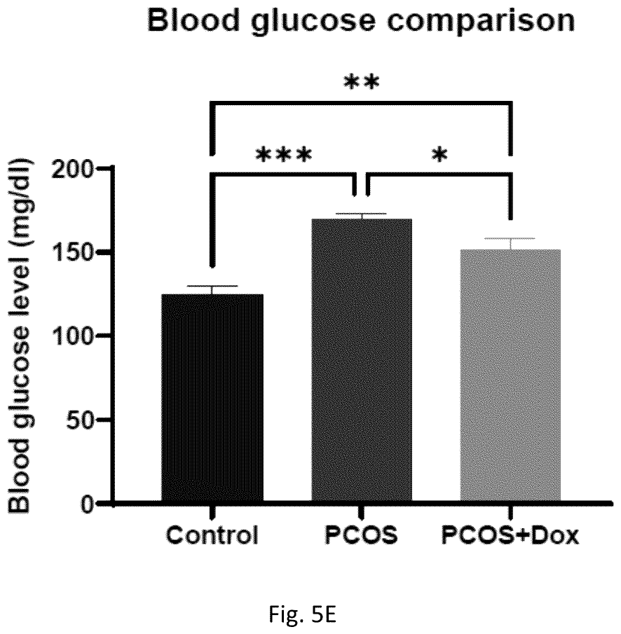

PCOS mice are treated with doxercalciferol to evaluate therapeutic potential according to the protocol in FIG. 5A. The PCOS mice gain more weight than control mice over the induction period (FIG. 5B) and doxercalciferol treatment lowers PCOS mouse weight during treatment (FIG. 5C). After the treatment is completed PCOS mice weigh significantly more than both control and doxercalciferol treated PCOS mice, and the control and doxercalciferol treated PCOS show no significant weight difference (FIG. 5D). Blood glucose levels are also assessed and PCOS mice have significantly increased levels compared to control. PCOS mice treated with doxercalciferol show significantly lower blood glucose levels than PCOS mice, but still have elevated levels compared to control mice (FIG. 5E). These results suggest that doxercalciferol treatment reverses obesity induced by PCOS.

### Effect of Doxercalciferol Treatment on PCOS Mouse Fertility

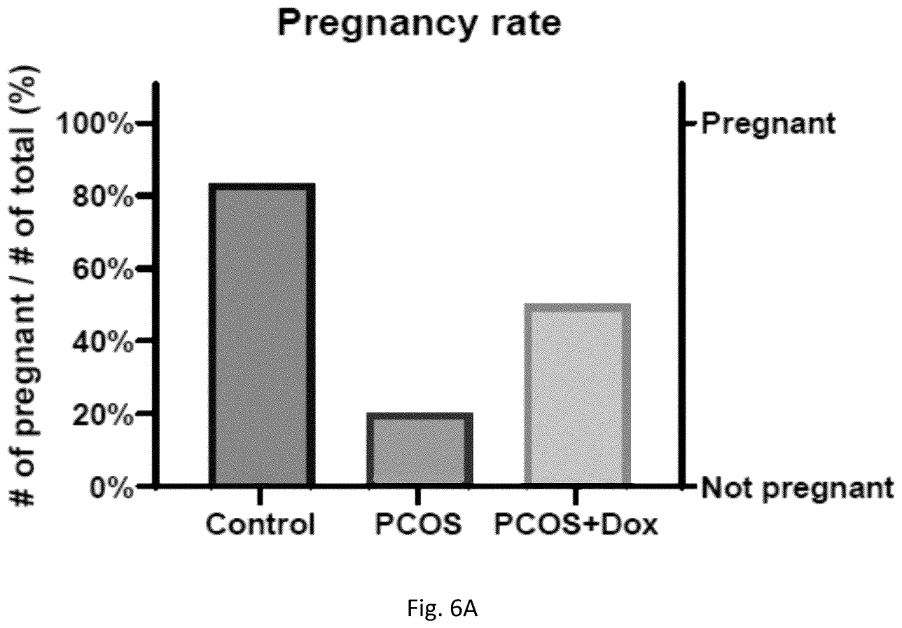

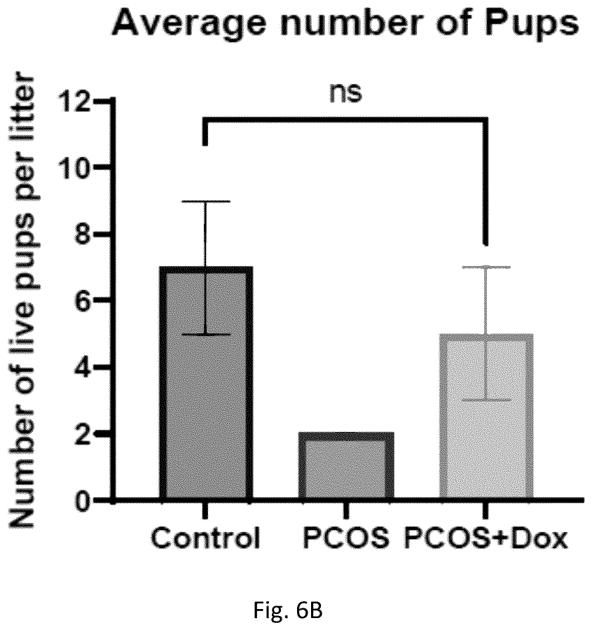

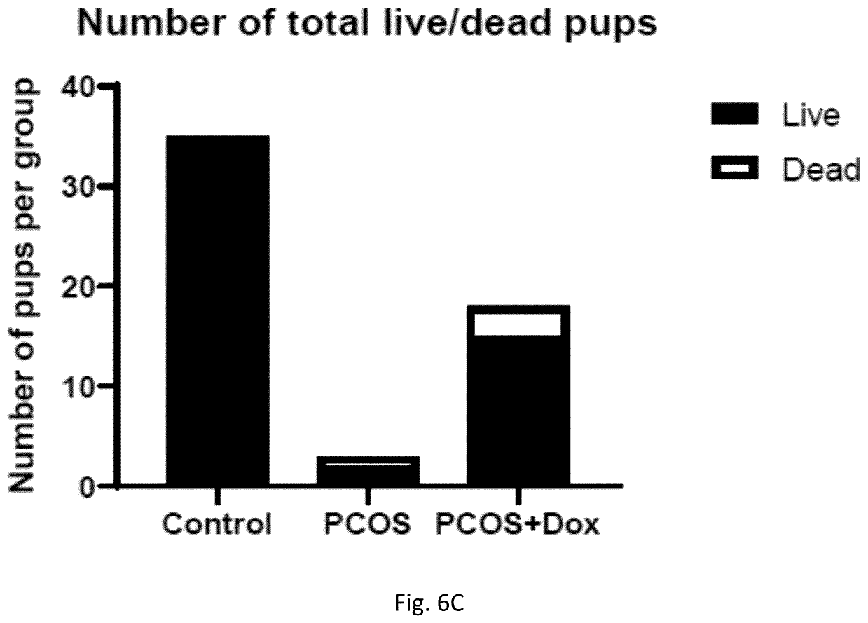

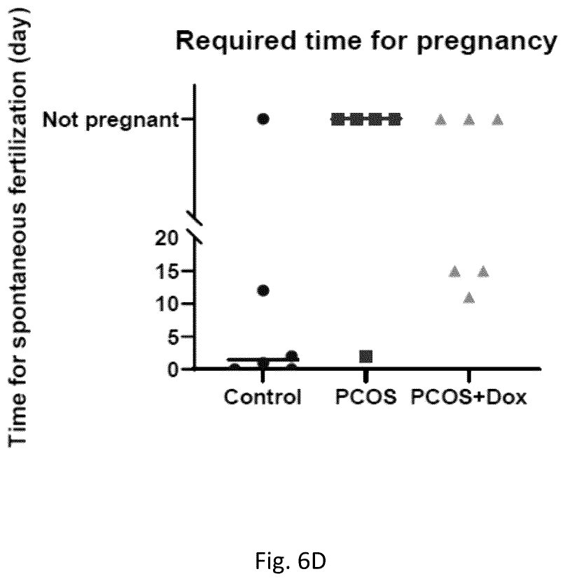

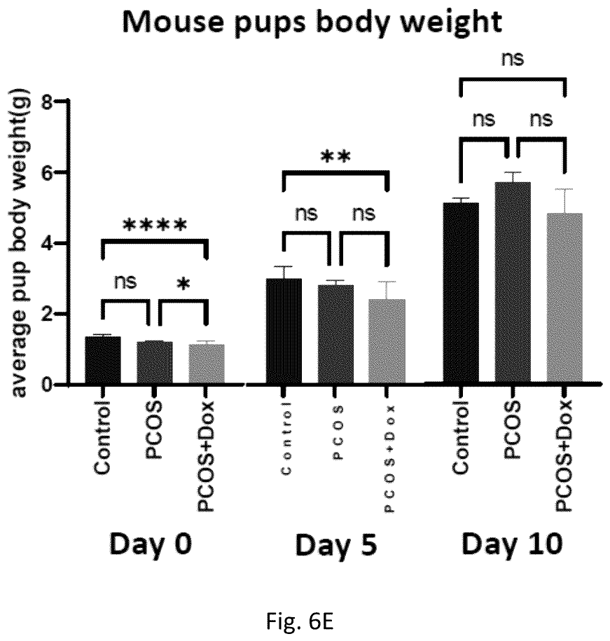

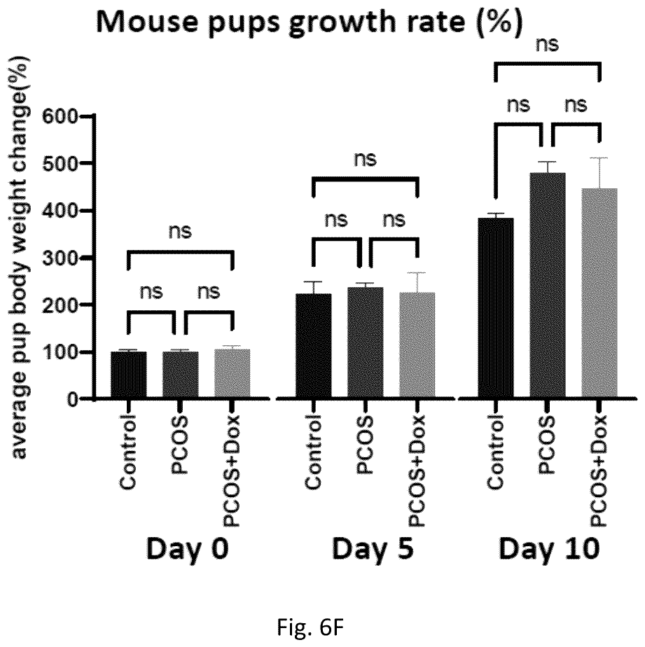

To further investigate the therapeutic potential of doxercalciferol for PCOS treatment its effect on fertility is assessed. The pregnancy rate in PCOS mice (20%) is as expected, much lower than in control mice (83.3%). Doxercalciferol treatment seems to partially rescue PCOS-related infertility by increasing the pregnancy rate to 50% (FIG. 6A). Doxercalciferol treatment of PCOS mice also increased the average number of live pups compared to untreated PCOS, while doxercalciferol treated PCOS mice show no significant difference to control mice (FIG. 6B). Doxercalciferol treatment also increases the total number of live pups born in PCOS mice compared to untreated, but not to the levels of control mice (FIG. 6C). While most control mice became pregnant with a few days of introduction to a potential mate, the doxercalciferol treated mice take about 2 weeks to become pregnant after introduction to a potential mate (FIG. 6D). The average weight of pups from doxercalciferol treated PCOS is significantly lower than control mice at birth and 5 day's postpartum, but at 10 days postpartum there is no significant difference in weight among pups born to control, PCOS, or doxercalciferol treated PCOS mice (FIG. 6E). There is also no significant difference in growth of pups from the control, PCOS, and doxercalciferol treated PCOS groups at birth, 5 days postpartum, or 10 days postpartum (FIG. 6F). These results suggest that doxercalciferol treatment at least partially rescues PCOS-related infertility while not adversely affecting the growth of offspring.

### Effect of Doxercalciferol Treatment on PCOS Mouse Tissue

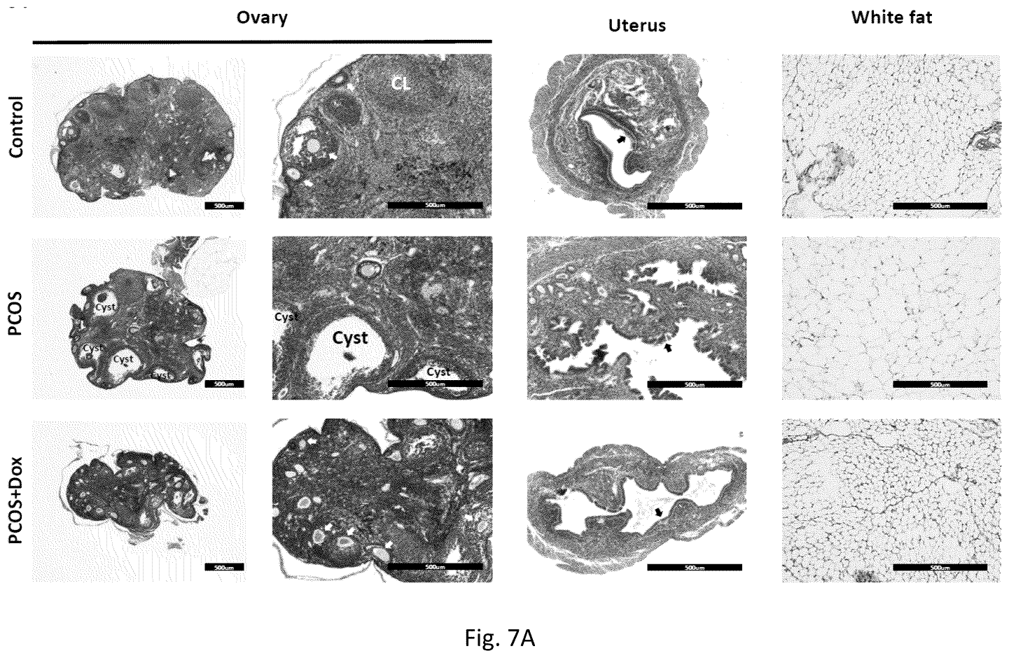

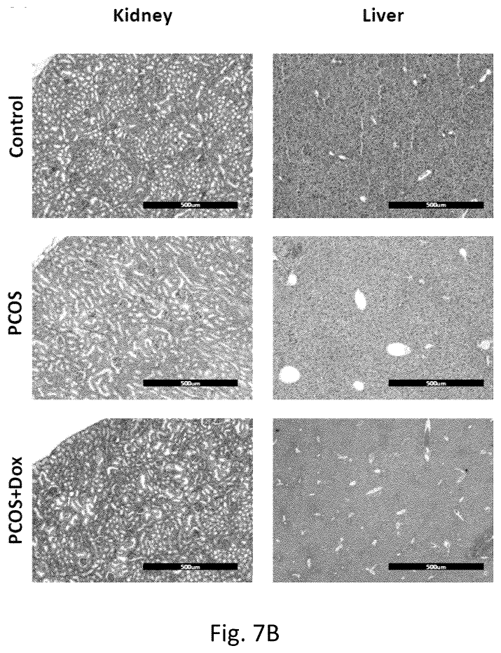

To further investigate the therapeutic potential of doxercalciferol for PCOS treatment its effect on PCOS-related tissues is assessed. After five weeks of treatment mice that had not been impregnated are euthanized and tissue from ovary, uterus, gonad white fat, kidney, and liver is removed and stained for imaging. The doxercalciferol treatment of PCOS mice show good numbers of follicles in the ovary while untreated PCOS mice have numerous cysts. Doxercalciferol treatment of PCOS mice also show improved uterine epithelial cell organization and reduced adipocyte size relative to untreated PCOS mice (FIG. 7A). There is no significant evidence of tissue damage or inflammation in the kidney or liver tissue of the control, PCOS, or doxercalciferol treated PCOS mice (FIG. 7B). These results suggest that doxercalciferol treatment inhibits ovarian cyst formation, uterine epithelial cell disorganization, and adipocyte hypertrophy.

### Effect of Doxercalciferol Treatment on PCOS Mouse Serum Chemistry

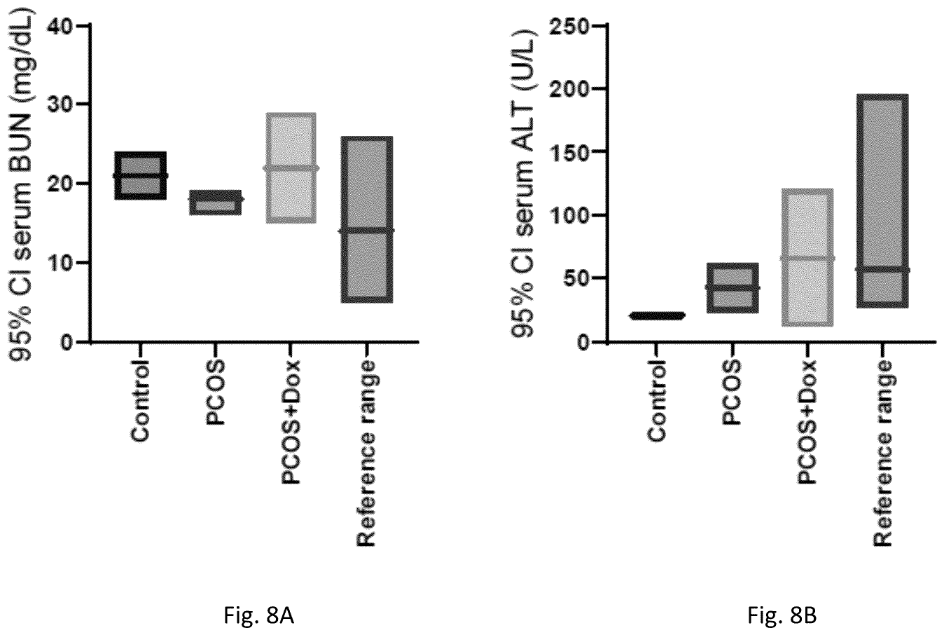

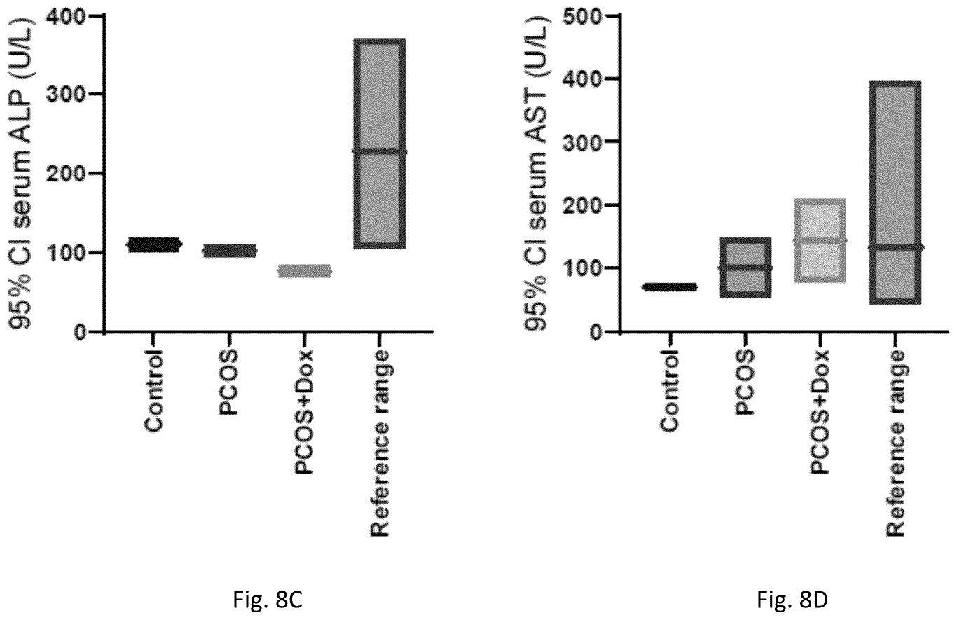

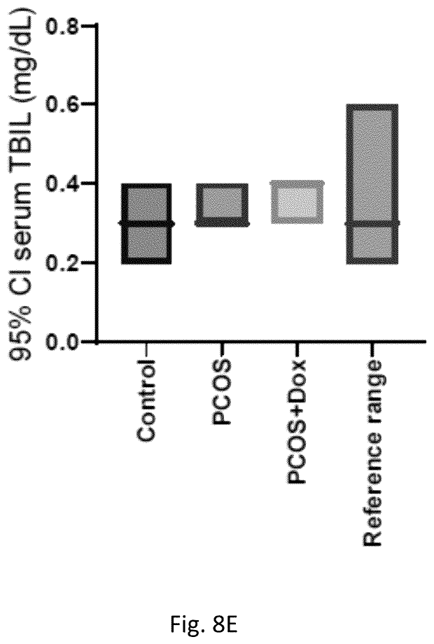

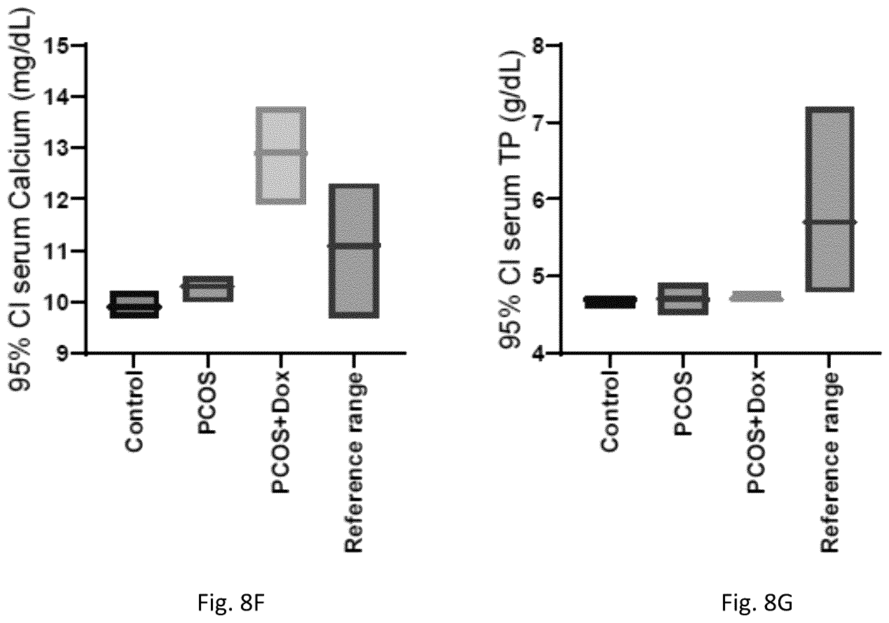

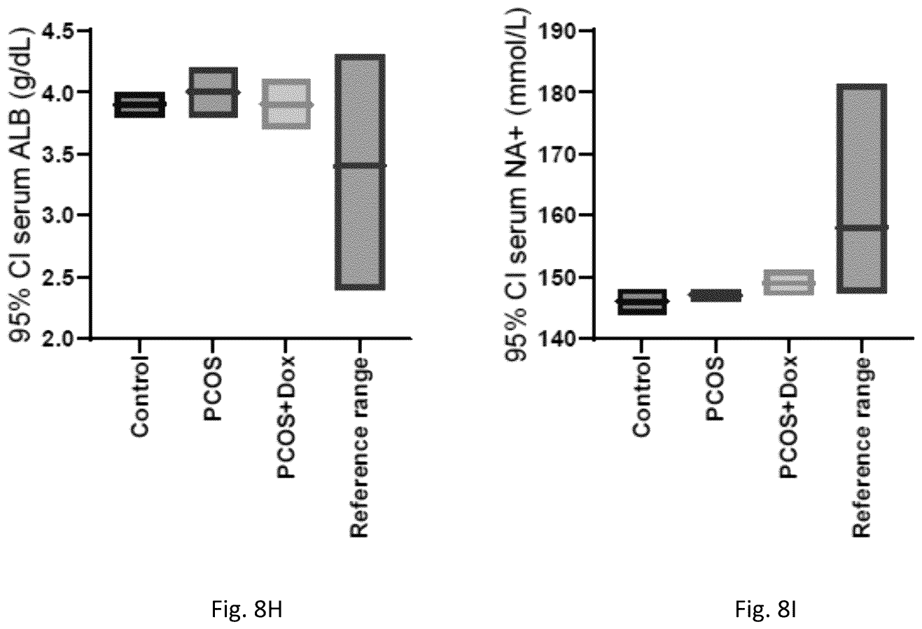

To further investigate the therapeutic potential of doxercalciferol for PCOS treatment its effect on blood serum levels is measured. After five weeks of treatment serum chemistry analysis is performed on control, PCOS, and doxercalciferol treated PCOS mice and compared with reference ranges for C57BL/6 mice. Serum chemistry analysis is performed for BUN (FIG. 8A), ALT (FIG. 8B), ALP (FIG. 8C), AST (FIG. 8D), TBIL (FIG. 8E), Calcium (FIG. 8F), TP (FIG. 8G), ALB (FIG. 8H), and NA+ (FIG. 8I). Most of the serum levels of control, PCOS, and doxercalciferol treated PCOS mice overlap with the reference range of C57BL/6 mice as shown in Table 1. These results suggest that doxercalciferol treatment does not adversely affect blood serum levels.

These results suggest that doxercalciferol would be an effective therapeutic for treating PCOS and at least reduce symptoms of weight gain, insulin resistance, infertility, and ovarian cyst formation, without adversely effecting subject blood serum levels or offspring growth.

TABLE 1

| | | BUN | ALT | ALP | AST | TBIL | CA | TP | ALB | NA+ |
|---|---|---|---|---|---|---|---|---|---|---|
| Mouse serum chemistry | | (mg/dL) | (U/L) | (U/L) | (U/L) | (mg/dL) | (mg/dL) | (g/dL) | (g/dL) | (mmol/L) |
| Reference range of | Mean | 14 | 57 | 228 | 133 | 0.3 | 11.1 | 5.7 | 3.4 | 158 |
| C57BL/6 strain | | | | | | | | | | |
| 95% CI | High | 26 | 195 | 370 | 397 | 0.6 | 12.3 | 7.2 | 4.3 | 181.2 |
| | Low | 5 | 27 | 105 | 43 | 0.2 | 9.7 | 4.8 | 2.4 | 147.5 |
| | n | 159 | 158 | 160 | 161 | 156 | 155 | 159 | 157 | 66 |
| control | Mean | 21 | 21 | 110 | 70 | 0.3 | 9.9 | 4.7 | 3.9 | 146 |
| | High | 24 | 23 | 119 | 75 | 0.4 | 10.2 | 4.7 | 4 | 148 |
| | Low | 18 | 18 | 101 | 64 | 0.2 | 9.7 | 4.6 | 3.8 | 144 |
| | n | 8 | 8 | 8 | 8 | 8 | 8 | 8 | 8 | 8 |
| PCOS | Mean | 18 | 43 | 102 | 101 | 0.3 | 10.3 | 4.7 | 4 | 147 |
| | High | 19 | 62 | 110 | 147 | 0.4 | 10.5 | 4.9 | 4.2 | 148 |
| | Low | 16 | 23 | 94 | 55 | 0.3 | 10 | 4.5 | 3.8 | 146 |
| | n | 8 | 8 | 8 | 8 | 8 | 8 | 8 | 8 | 8 |
| PCOS + Dox | Mean | 22 | 66 | 76 | 144 | 0.4 | 12.9 | 4.7 | 3.9 | 149 |
| | High | 29 | 120 | 84 | 209 | 0.4 | 13.8 | 4.8 | 4.1 | 151 |
| | Low | 15 | 12 | 68 | 79 | 0.3 | 11.9 | 4.7 | 3.7 | 147 |
| | n | 7 | 7 | 7 | 7 | 7 | 7 | 7 | 7 | 7 |

### Example 4

### Materials and Methods

### Patient Derived Xenograft (PDX) Mouse Model of UF Doxercalciferol Treatment

Female NOD/SCID are housed in a vivarium for 1 week under specific pathogen-free conditions. The animal experiment protocol for this study is approved, and all animal experiments are performed in compliance with policies and guidelines for use of laboratory animals.

UF samples are obtained from African-American female patients with UF (n=4). This human UF tissue is xenografted into female mice that have been hormonally supplemented with progesterone and estradiol (n=2 mice/patient). Tumor size is measured at implantation.

After xenograft mice undergo intraperitoneal injection of doxercalciferol (300 ng/kg, 3 times per week) or saline for 6 weeks. Body weight is monitored weekly during doxercalciferol treatment. Six weeks after doxercalciferol treatment insulin resistance is assessed using GTT and then mice are anesthetized and blood is collected to measure liver enzyme and AMH levels, ovary tissue is removed for histological analysis, total RNA is extracted for sequencing, and tumor size is measured with a digital caliper.

### Glucose Tolerance Test

Glucose tolerance testing is performed on PDX UF mice 6 weeks after doxercalciferol treatment. Mice are tested either without fasting or after fasting for 16 h (5 p.m. to 9 a.m.), with free access to drinking water, after which they receive an intra-peritoneal (i.p.) injection of D-glucose (2.0 g/kg body weight). Blood glucose level is measured at 0, 15, 30, 60, 90, and 120 min following glucose injection using a Bayer glucose monitor (Roche Diagnostics Corp, IN, USA).

### Differentially Expressed Gene (DEG) Analysis

Total RNA is extracted from PDX mice after doxercalciferol treatment. EdgeR is used to identify DEGs with p-values of (p)<0.01). GO and KEGG enrichment analysis are performed to determine the implication of the DEGs at the biologically functional level.

### Serum Measurements

Blood is collected from all groups by cardiac exsanguination under isoflurane anesthesia: serum is separated and stored at −80° C. Serum levels are measured for AMH, ALT, and total bilirubin.

### Histology and Immunohistochemistry

Ovary tissue is collected and fixed in 4% paraformaldehyde. Tissue sections are stained with hematoxylin-eosin (H&E). Histological analyses are performed.

### Statistical Analysis

Comparisons between groups are made as described in example 1.

### Results

### Effect of Doxercalciferol Treatment on Patient Derived Xenograft (PDX) Mouse Model of Uterine Fibroid (UF) Tumor Growth, Body Weight, and Glucose Levels

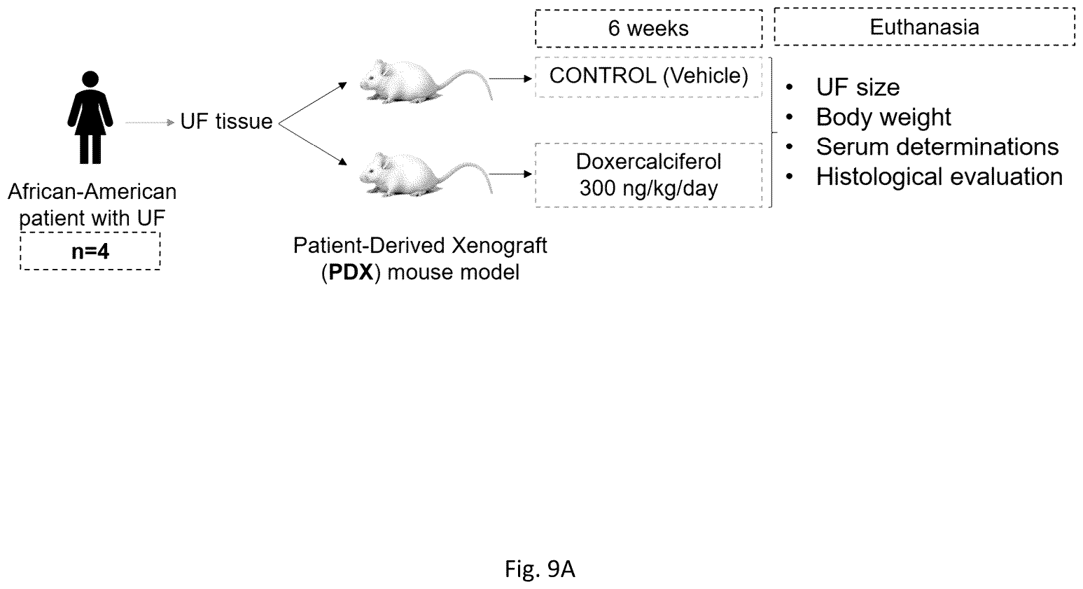

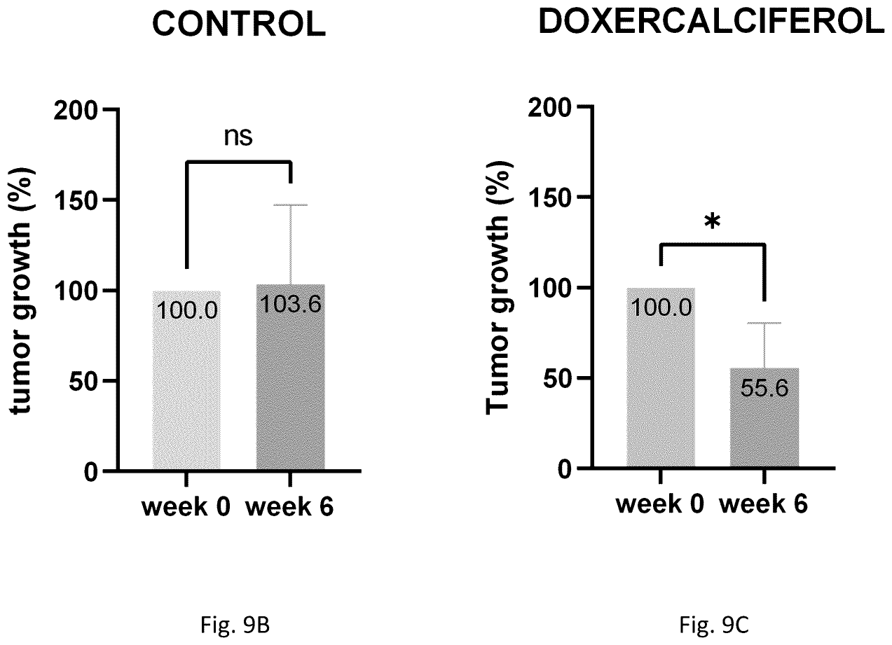

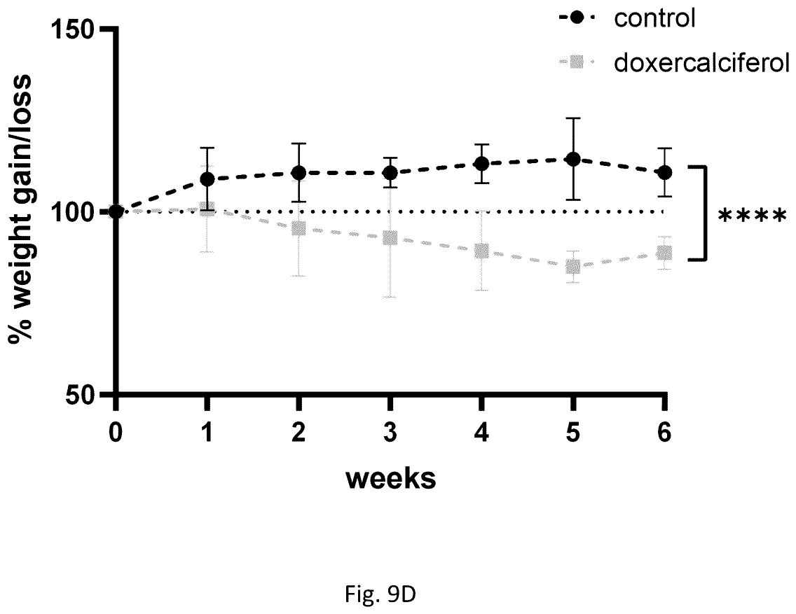

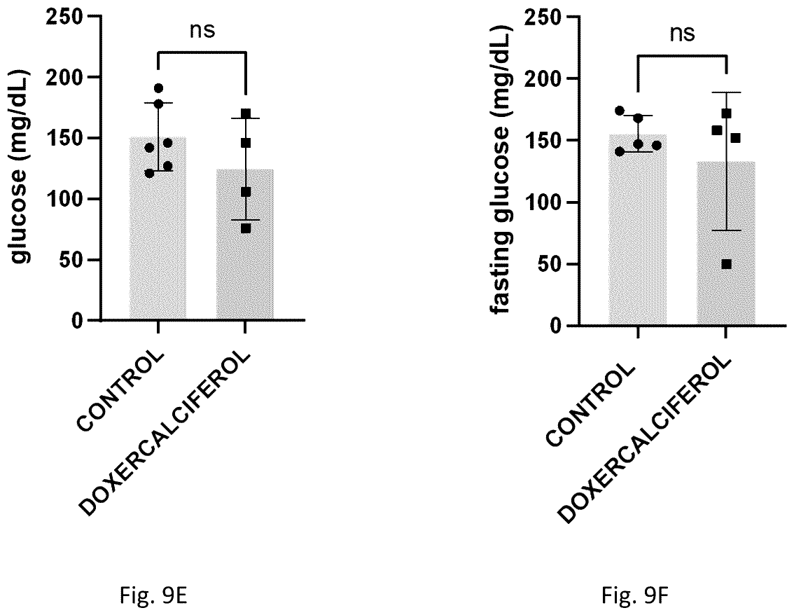

PDX mice are treated with doxercalciferol to evaluate therapeutic potential according to the protocol in FIG. 9A. The tumor growth rate is measured between the xenograft and the end of treatment, 6 weeks later. Control PDX mice show no significant difference (FIG. 9B), while doxercalciferol treated PDX mice show a significant reduction in tumor growth, down to 55.6% (FIG. 9C). The rate of weight change is also assessed during the 6 week treatment period and differ significantly between control PDX mice and doxercalciferol treated PDX mice. Doxercalciferol treatment induces a decrease of 24% in body weight of PDX mice (FIG. 9D). Doxercalciferol treatment also tends to lower both glucose and fasting glucose levels in PDX mice (FIGS. 9E and 9F). These results suggest that doxercalciferol inhibits both UF tumor growth and UF related weight gain.

### Effect of Doxercalciferol Treatment on PDX Mouse Model Differentially Expressed Genes (DEGs)

To further investigate the therapeutic potential of doxercalciferol for UF treatment its effect on gene expression is assessed. RNA sequencing analysis reveals 410 DEGs (158 up- and 252 down-regulated) in UFs treated with doxercalciferol compared to control. Among the top 20 DEGs, the treatment significantly upregulates DUSP1 (fold change [FC]=1.05), involved in negative regulation of cell proliferation, and IL24 (FC=1.55), which has anti-proliferative and pro-apoptotic properties in cancer. Doxercalciferol also downregulates the expression of ARGI (FC=−1.42), involved in collagen synthesis, and the proto-oncogene ADRA1B (FC=−1.81). GO analysis shows significant enrichment in extracellular matrix (ECM), collagen-containing ECM, and ECM organization, among others. KEGG enrichment analysis shows an over-representation of cytokine-cytokine receptor interaction, ECM-receptor interaction, and retinol metabolism. These results suggest that doxercalciferol treatment alters gene expression to upregulate genes related to inhibiting tumor growth and downregulate genes related to tumor growth, particularly ECM synthesis.

### Effect of Doxercalciferol Treatment on PDX Mouse Model of UF Tissue and Serum Levels

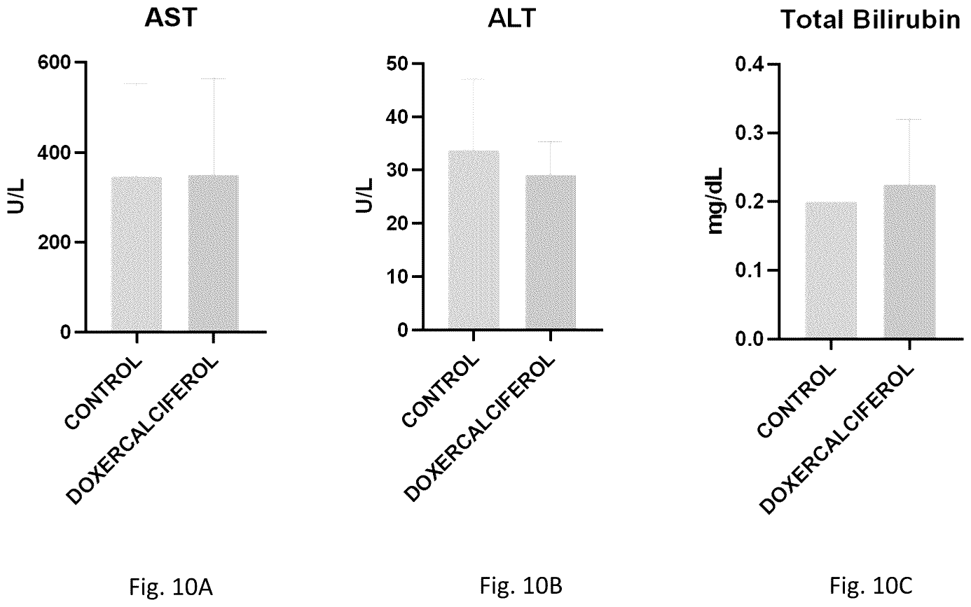

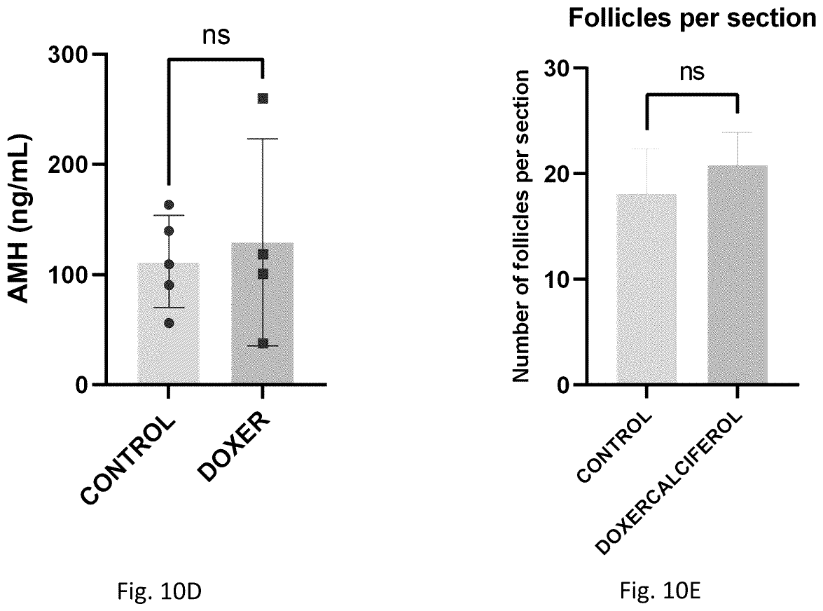

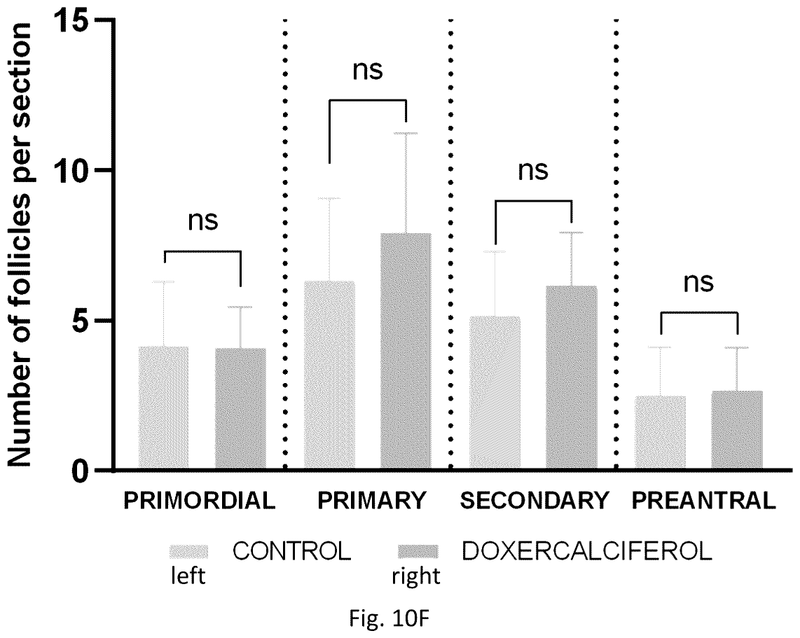

To investigate the safety of doxercalciferol for UF treatment its effect on UF related tissues and serum levels is measured. Doxercalciferol treatment has no significant effect on levels of the liver enzymes AST, ALT, or total bilirubin levels of PDX mice (FIGS. 10A-10C). The calcium levels in both doxercalciferol treated and untreated PDX mice overlap with the reference range for NOD CB17-Prkdc^{scid}/NcrCrl mice (Table 2). Doxercalciferol treatment of PDX mice also has no significant effect on AMH serum levels (FIG. 10D). Doxercalciferol treatment of PDX mice does not significantly affect either the total number of follicles or the number of follicles at the primordial, primary, secondary, or preantral phases in the ovary relative to untreated control PDX mice (FIGS. 10E-10F). These results suggest that doxercalciferol treatment of UF neither adversely affects liver enzyme, calcium, or serum levels nor folliculogenesis.

These results suggest that doxercalciferol would be an effective therapeutic for treating UF and at least reduce tumor growth and weight gain, without adversely affecting subject liver enzyme, calcium, and AMH levels or folliculogenesis.

TABLE 2

| | CONTROL | DOXERCALCIFEROL | REFERENCE |
|---|---|---|---|
| Minimum | 8.5 | 9.7 | 10.2 |
| Maximum | 10.9 | 13.7 | 11.8 |
| Range | 2.4 | 4 | 1.6 |
| Mean | 9.533 | 11.93 | 11 |
| Std. Deviation | 0.9933 | 1.658 | — |
| Std. Error of | 0.4055 | 0.829 | — |
| Mean | | | |
| N | 6 | 4 | 63 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing uterine fibroids (UF) or polycystic ovary syndrome (PCOS) in a female mammal, the method comprising administering to the female mammal an effective amount of doxercalciferol.

2. The method of claim 1, wherein the method is a method of treating or preventing UF.

3. The method of claim 1, wherein the method is a method of treating or preventing PCOS.

4. The method of claim 3, wherein the method is a method of treating or preventing PCOS-related insulin resistance.

5. The method of claim 3, wherein the method is a method of treating or preventing PCOS-related infertility.

6. The method of claim 3, wherein the method is a method of treating or preventing PCOS-related obesity.

7. The method of claim 3, wherein the method is a method of treating or preventing PCOS-related hyperandrogenism.

8. The method of claim 2, wherein the method is a method of treating UF.

9. The method of claim 3, wherein the method is a method of treating PCOS.

10. The method of claim 8, wherein the administration is intravenous.

11. The method of claim 8, wherein the administration is oral.

12. The method of claim 8, wherein the female mammal is human.

13. The method of claim 12, wherein the effective amount of doxercalciferol is 4000 IU administered 3 times a week for 12 weeks.

14. The method of claim 9, wherein the administration is intravenous.

15. The method of claim 9, wherein the administration is oral.

16. The method of claim 9, wherein the female mammal is human.

17. The method of claim 16, wherein the effective amount of doxercalciferol is 4000 IU administered 3 times a week for 12 weeks.
